(19) United States
(12) Patent Application Publication
(10) Pub. No.: US 2018/0250238 A1
(43) Pub. Date: Sep. 6, 2018

(54) SOLID DRUG DISPENSING APPARATUS, FORMULATIONS, AND METHODS OF USE

(71) Applicant: InCube Labs, LLC, San Jose, CA (US)

(72) Inventors: ELMAR FISCHER, BOERN, TX (US); PAUL SPEHR, SAN ANTONIO, TX (US); BRUCE ANDERSON, SAN ANTONIO, TX (US); MIR IMRAN, LOS ALTOS HILLS, CA (US)

(73) Assignee: InCube Labs, LLC, San Jose, CA (US)

(21) Appl. No.: 15/904,207

(22) Filed: Feb. 23, 2018

Related U.S. Application Data
(60) Provisional application No. 62/462,855, filed on Feb. 23, 2017.

(51) Int. Cl. A61K 9/70; A61M 37/00; A61M 31/00; A61K 31/635; A61K 31/167; A61K 31/46; A61K 31/4458; A61K 31/04; A61K 31/4745; A61K 38/28; A61K 38/22; A61K 47/10

(52) U.S. Cl. CPC A61K 9/70; A61M 37/0069; A61M 31/002; A61K 31/635; A61K 31/167; A61K 31/46; A61M 25/0068; A61K 31/04; A61K 31/4745; A61K 38/28; A61K 38/22; A61K 47/10; A61K 31/4458

(57) ABSTRACT

Embodiments provide apparatus and methods for delivering solid form medications such as drug cords (DC) to various locations in the body. The DCs may comprise one or more drugs and excipients. One embodiment provides an apparatus for in vivo delivery of DCs comprising a housing including a port, a DC disposed in the housing on a spool or like device and a mechanism for advancing the DCs from the inside the housing to a delivery site (DS) outside the housing. The apparatus can be used to deliver selected lengths of DC corresponding to a dose of drug to treat a medical condition. The DC can be delivered to the DS at regular intervals or responsive to a detected biological event. Embodiments of the invention are particularly useful for delivering medication to treat a selected medical condition over an extended period without requiring a patient to take external medication.

### RELATED APPLICATIONS

This application claims the benefit of priority of U.S. Provisional Application No. 62/462,855, entitled “Solid Drug Dispensing Apparatus, Formulations And Methods Of Use”, filed Feb. 23, 2017; which is fully incorporated by reference herein for all purposes.

The disclosure of this application is also related to those of U.S. Provisional Patent Application Ser. Nos. 61/210,579 and 61/210,554, both filed Mar. 20, 2009 and both entitled “Solid Drug Delivery Apparatus and Formulations and Methods of Use” and U.S. patent application Ser. No. 13/138,764, 371e date, Jul. 13, 2012, entitled “Solid Drug Delivery Apparatus and Formulations and Methods of Use all of which are all incorporated by reference herein in their entirety for all purposes.

### BACKGROUND OF THE INVENTION

### Field of the Invention

Embodiments of the invention relate to drug delivery devices and methods for their use. More specifically, embodiments of the invention relate to implantable drug delivery devices for the delivery of solid form drugs and other therapeutic agents.

The current trend in many medical treatments requires the delivery of a drug to a specific target site so as to avoid the toxicity to other tissue, as well as more precisely controlling the timing and amount of drug delivered to that site. In many cases, this can require an implantable drug pump. However, due to their size and power requirements, the uses of currently available drug pumps have been limited, rarely being used for delivery of medications to the brain, heart and other tissues, where very precisely controlled doses of drug can be required. Also current devices can require frequent replenishment of the drug due to limited reservoir size and/or limited shelf life of the drug. Thus, there is a need for improved implantable drug delivery devices and associated methods for in vivo drug delivery.

### Description of the Background Art

Drug delivery apparatus employing rotatable spools are described in U.S. patent application Ser. Nos. 14/905,080 and 14/783,496 which are incorporated by reference herein for all purposes

### BRIEF SUMMARY OF THE INVENTION

Embodiments of the invention provide apparatus, systems, formulations and methods for delivering medications in solid form to various locations in the body of a human patient or mammal. Many embodiments provide an implantable apparatus for delivering medication or medicaments in solid form wherein the medication includes one or more solid form drugs or other therapeutic agents for treating various medical conditions such as epilepsy and other neural condition; diabetes and other endocrine conditions; and cardiac arrhythmias and other cardiac rhythm disorders. Particular embodiments provide an enclosed implanted apparatus for delivering solid form medications in the form of a cord or string to a delivery site so as to treat a medical condition for an extended period of time. Embodiments also provide various solid form medicament preparations comprising one or medicaments to be delivered by embodiments of the apparatus or other drug delivery apparatus. Such medicaments including any drug, active substance, biological substance, polypeptide, small molecule protein, or antibody or the like that can be beneficially administered to the patient at a tissue target site whether for therapeutic or diagnostic purposes.

Particular embodiments provide novel implantable drug storage and dispensing devices which dispense drug doses using a medicament preparation in the form of a long thin flexible cord or thread which may be stored on a spool or other similar storage means. For ease of discussion, the medicament preparation is herein described as a drug cord, though other forms described herein are equally applicable. In one or more more embodiments, the drug cord or portions thereof such as a core portion may have similar material properties as fishing line in that it is flexible enough to be bent in a tight spool, yet it is stiff and rigid enough to be grasped between compression wheels or a similar mechanism so as to be pushed down a catheter or like device to a selected drug delivery site in the body of the patient or mammal. In particular embodiments, the drug cord may be stored in a coil wound onto a spool in the drug storage device to minimize storage volume. This drug dispensing device has an electro-mechanical linear drive system means of pushing the drug cord through a catheter lumen to deliver the drug cord to the tip of this catheter. The lumen of this catheter will have one or more seals with respect to the drug cord to minimize moisture ingress via the catheter lumen to allow moisture inside the drug storage device. The drug cord may exit the catheter lumen directly into a target tissue or intravascular site, but will more often exit the catheter lumen into a diffusion chamber at the catheter tip. Body fluids can flow into the diffusion chamber to dissolve/absorb the exposed drug cord. The drug delivery can occur periodically, based on a manual command, and/or based on sensed physiological signal(s) from the body (e.g., high blood glucose). The implantable device can optionally contain physiological sensor(s) and a microcontroller that could together detect biological need (e.g., high blood glucose) and generate a command for dispensing and delivering drug based on the detected biological need.

The drug may be contained in the flexible cord in a variety of forms. For example, according to one or more embodiments the drug cord may comprise a solid core with or without an overlying layer of a biodegradable polymer containing the drug in a dispersed form, in another embodiment the entire cord may comprise a dispersion of drug in a biodegradable polymer also referred to herein as a matrix material. The cord will be maintained inside the implantable drug delivery device, typically within a hermetically sealed container with a sealing dispensing port for advancing the drug into a dispersion chamber and/or a tissue or vascular target site. The drug cord will often be stored on a spool which can be controllably rotated to control the dosage to be delivered. The drug cord is usually sufficiently flexible to allow reeling and unreeling from the spool but will have sufficient column strength so that it may be advanced by rollers or other mechanical advancement mechanisms. When advanced by opposed roller, the cord should have sufficient radial incompressibility to allow traction for movement.

In various embodiments, the catheter may have smooth inner lumen for passage of the drug cord. Or alternatively, the inner lumen of the catheter may be lined with a coil, which may allow for a lower sliding force. At least one seal, such as a lip seal, will be located in the catheter tip between the diffusion section and the drug storage. According to various embodiments, the tip of the catheter may include a diffusion chamber in which the drug cord is dissolved by bodily fluids such as blood or CSF (cerebral spinal fluid) which enter into the diffusion chamber. Body fluids will pass into the diffusion chamber and absorb/dissolve the drug\exposed cord. The tip of the catheter is positioned at the drug therapy site. The size of the spool holding the drug cord can be determined based on one or more of drug dosage, diameter of the drug cord, and drug cord length.

Embodiments of the invention contemplate at least two electro-mechanical methods for dispensing the drug cord out of the housing. In one approach, a drive system can include two opposed drive wheels which be used to engage and advance the drug cord. At least one drive wheel would be powered by an electrical motor, typically through a geared reducer drive system.

In a second approach for advancing the drug cord, the drive system includes a reciprocating, linear driver, e.g. a grabber, having flexible arms that press against the drug cord and are bent in a biased engagement with the drug cord. When the grabber is advanced, the drug cord is pushed forward by the arms from the grabber engaging the drug cord. When the motion of the grabber is retracted, the grabber disengages the drug cord in a ratcheting-type movement. The grabber may be driven, for example, using a geared electric motor with a screw-drive connected directly to the grabber. There could be one more sensors embedded in the device to sense when the grabber is fully retracted and ready for the next drug delivery.

According to various embodiments, the ratio of the drug cord width (diameter) to the inner spool height can be selected to minimize tangling. In various embodiments the ratio can be in the range of about 1:4, 1:5, :1:7, 1:8, 1:9, 1:10, 1:12, 1:15 and 1:20. In preferred embodiments, the ratio can be in the range of about 1:8 to 1:12. For example, the cord may have a mean width, typically a diameter, in the range from 1 mm to 20 mm, typically from 5 mm to 15 mm, and the spool may have an inner diameter (diameter of the surface of the spool upon which the flexible drug cord is wound) in the range from 8 mm to 200 mm, typically from 20 mm to 100 mm. The length of the flexible drug cord can vary widely, typically being at least 10 cm, often being at least 20 cm, and frequently being 30 cm or longer. Typical ranges of length are from 5 cm to 30 cm, often from 10 cm to 40 cm.

In various applications, embodiments of the invention can be used to deliver solid form drugs to different locations in the body for treatment for a number of medical conditions including, for example, to the heart for treatment of coronary arrhythmia's (both atrial and ventricular), coronary ischemia (e.g., from a heart attack and/or stenosed coronary artery); the brain for the treatment of epilepsy, brain tumors such as malignant gliomas, cerebral ischemia, stroke; or to the arterial or venous vascular system for treatment of one or more of high blood pressure, anemia, diabetes infection or septicemia or cancers such as leukemia. The apparatus can be implanted at or near the target tissue site (e.g., the brain) or at remote delivery site (e.g., sub-dermally, intramuscularly in the chest or thigh). Further embodiments of the invention can be used to provide concurrent treatment for two or more of these or other conditions eliminating the need for the patient to take multiple doses of multiple drugs (e.g., orally or by parenteral means) over the course of day. This is particularly beneficial to patients who have long term chronic conditions including those who have impaired cognitive abilities.

In an exemplary embodiment of a method for using the invention, the apparatus can be implanted at a selected delivery site (e.g., the brain, or the pectoral region for drug delivery to the heart) depending on the condition to be treated. Implantation can be done using an open or minimally invasive surgical procedure known in the art. Prior to implantation, the apparatus can be loaded with a selected length of drug cord for delivery of a prescribed number of doses. Once implanted, the flexible drug cord can be stored in the apparatus for an extended period of time (e.g., six months, 1 year, 2 years, 5 years, or longer) without degradation or deleterious effect to the drug cords (e.g., loss of drug potency or therapeutic effectiveness) due to the fact that the drug cords are stored in the housing. The apparatus can deliver length of the flexible drug cord comprising a drug of interest to the target tissue site at regular intervals (e.g., once a day, week, month, etc.) or in response to an input from a sensor. In the latter case, the input can be in response to a particular medical condition or biological/physiological event, such as an epileptic seizure or pre-seizure event.

A controller described hereinafter can be used to determine when to initiate delivery based on the sensor input and/or the time intervals for delivery for embodiments employing delivery at regular intervals. In either case, the controller can send a signal to the mechanism to deliver a selected length of drug cord, corresponding to a selected drug dose, from the housing interior to the tissue site. There, the selected length of the drug cord disintegrates/degrades and is dissolved in local tissue fluids to treat a local target tissue site (e.g., it dissolves in the CSF to treat the brain for epilepsy), or it is subsequently absorbed into the blood stream where it is carried to a remote target tissue site (e.g., the liver, heart, etc.) or both. Optionally, the selected length of the drug cord is dissolved in a diffusion chamber prior to being released to the target tissue site. Still further optionally, the length of drug cord is advanced from the enclosure through a catheter to the diffusion chamber so that the target tissue site can be located remotely from the enclosure implantation site. Further, drug cord lengths (doses) can be delivered based on input from a sensor providing physiologic data predictive of the medical condition (e.g., blood glucose) or another sensor that is configured to sense the local and/or plasma concentration of the drug. In some embodiments, drug delivery can be controlled by sensing the state of disintegration of previously delivered cord length (i.e., drug dose). For example, another drug cord length can be delivered when it has been determined that the previous cord length is in a particular state of disintegration (e.g., it has been completely or substantially disintegrated). This can be achieved by sending and receiving a signal from the dispensed drug cord length such as an optical, ultrasound or electrical signal. For example, for the use of optical signal reflectance, measurements can be used to determine the state of disintegration. A particular disintegration state can be determined when the reflectance signal falls below a particular threshold. Similar approaches can be used for use of reflected ultrasound or impedance. According to some embodiments, the drug cord can even include various echogenic, or optically opaque or other agents to enhance the reflected ultrasonic, optical or other signal. The drug cord may also include various optical indicia having one or more of a pattern, size or shape configured to provide an indication of the state of disintegration of a dispensed length of drug cord.

In a first exemplary embodiment, the present invention provides an apparatus for in vivo delivery of various solid forms of medication to a patient. The apparatus comprises a housing or enclosure configured to be implanted within a body of the patient where the housing will have a least one wall enclosing an interior volume. While the housing will have at least one aperture or port for releasing the flexible drug cord, as discussed in detail below, it will otherwise be sealed, preferably hermetically sealed, to inhibit or prevent the intrusion of body fluids, moisture, and other deleterious materials so that such materials cannot pass into the interior of the housing after the housing has been implanted in the patient. As discussed in more detail below, the at least one port or aperture through the wall of the housing or enclosure will be provided with seals which inhibit or prevent the passage of fluids into the interior of the housing as the flexible drug cord is being advanced out of the housing. The housing or enclosure could have additional ports, apertures, or other passages formed through the wall for other purposes, such as electrical conduits or connectors, but such additional passages will usually also be sealed to protect the interior from unwanted intrusions.

The drug delivery apparatus of the present invention will usually further comprises a spool rotatably mounted within the interior of the housing and configured to receive a continuous length of a flexible drug cord wound over a surface of the spool, typically over an inner core of the spool. The cord may wound over the spool under tension as well to keep the cord from tangling. The flexible drug cord will be disposed and configured to be advanced out through a port in the housing wall, and a flexible seal on the port is figured to seal over the drug cord as the drug cord is advanced through the port. The drug cord advancement mechanism is configured to advance the drug cord through the port and out of the housing. A controller, usually mounted within the housing or enclosure, is configured to actuate the drug cord advancement mechanism to advance a length of the drug cord out of the housing in response to an input. Typically, a battery or other power source will also be mounted within the interior. The battery may be single-use or rechargeable, typically being wirelessly rechargeable via an RF link.

In exemplary embodiments, the apparatus will further comprise a diffusion chamber configured to receive a dispensed length of the drug cord and expose the dispensed drug cord length to body fluids to dissolve the drug cord length and allow a solution comprising the drug to diffuse out of the diffusion chamber. Usually, the apparatus will still further comprise cutting means associated with the housing or enclosure and configured to cut the drug cord after it has been dispensed out of the housing and usually into the diffusion chamber. For example, the cutting means may comprise one of a blade, an ultrasonic cutter, a heated cutting element, or the like. In other embodiments, the drug cord may be partially pre-cut (or otherwise scored, notched or shaped or treated to facilitate separation) at set lengths corresponding to a desired dosage. When partially pre-cut, separation can be effected by still other means, such as applying tension.

Exemplary drug cord advancement mechanisms will typically comprise driver elements which engage and push and/or pull the drug cord in a motor coupled to drive the drive elements. In the first exemplary embodiment, the driver elements may comprise a pair of opposed pinch rollers. In a second specific embodiment, the driver elements may comprise a reciprocating linear advancement mechanism.

In still other specific embodiments, the apparatus of the present invention may comprise a catheter coupled to the port on the housing, where the catheter is configured to guide the flexible drug cord to the diffusion chamber. Usually, the diffusion chamber will be coupled to a distal portion of the catheter, and the cutting means will most often be mounted within the diffusion chamber at the end of the catheter. In other specific features, the apparatus of the present invention may have an atraumatic distal end to allow for extended periods of implantation at the implantation site, and at least a portion of apparatus may be coated with a biocompatible coating. Suitable biocompatible coatings include silicones, polyurethanes, fluoropolymers, PARYLENE and the like.

Embodiments of the apparatus controller may be programmed in various ways to perform various functions. For example, it may be programmed and configured to deliver a dose of medication continuously or at intervals, typically regular intervals. Alternatively, the controller may be configured to respond to an externally transmitted signal (e.g., radio frequency (RF), magnetic, ultrasonic, or other like signal) in order to initiate delivery and/or reprogram a delivery pattern. Still further alternatively, the controller may be programmed and configured to respond to sensor(s) located within or externally to the patient, where the sensors can detect physiological events to trigger a drug release. Exemplary physiological events may include epileptic seizures, pre-seizure events, cardiac arrhythmias, hyperglycemia, hypertension, and the like.

In a second exemplary aspect of the present invention, a flexible drug cord may comprise a matrix material formed into an elongate flexible cord, where the matrix material is degradable in a physiologic environment (e.g. such as in the CSF of the brain or blood in the heart) and desirably non-degradable in a hermetically sealed environment. The matrix material may comprise various biodegradable polymers known in the art, a preferred example include polyethylene oxide (PEO). At least one drug will be disbursed or otherwise distributed within at least a portion of a length of the matrix material. The matrix material formed with the at least one drug is desirably configured to be (i) wound and stored on a spool in a housing implanted within the patient's prosperous body for an extended period without substantial degradation or deleterious effect to the medication, (ii) delivered to a delivery site by unwinding from the spool and advancing through a port in the housing, and (iii) dissolved in tissue fluids at a target tissue site to release the drug and produce a therapeutic affect at the target tissue site to treat a disease or condition.

According to one or more embodiments, the flexible drug cord may comprise a drug which may include a therapeutic or diagnostic agent. Suitable drugs may include a drug for the treatment of epilepsy, such as furosemide; a drug for the treatment of an arrhythmia such as lidocaine atropine or flecainide (e.g., Flecainide Acetate; a drug for the treatment of diabetes; a drug for the treatment of angina; a drug for the treatment of cancer such as topotecan for the treatment of glioblastomas and other brain cancer. According to various embodiments the drug cord further may comprise multiple drugs in a single drug cord for the treatment of a single or multiple diseases or other medical conditions. Tissue target sites will typically be in solid tissue, such as muscular tissue, adipose tissue, cerebral tissue, solid organ tissue, or the like. Optionally, in addition to the matrix material, the drug cord may comprise a pharmaceutical excipient intended to interact with the drug in a beneficial or synergistic manner. The drug cords may further comprise, in addition to the matrix material and the excipient, further components providing or acting as a disintegrant (including super disintegrants) or a preservative or stiffening agent.

In a third exemplary aspect, the present invention provides a method for delivering a drug to a patient using a drug cord. The method comprises implanting an enclosure or housing containing an elongate flexible drug cord wound on a spool in the body of the patient for an extended period of time without substantial degradation of, or deleterious effect to, the medication. The spool is unwound allowing the flexible drug cord to advance through a port or opening on a wall of the enclosure and to a tissue target site in the patient's body. The length of the flexible drug cord which is released degrades in or near the tissue target site to release the drug at the tissue target site to produce a therapeutic effect for the treatment of a disease or condition.

In specific examples, unwinding the spool to advance a length of the flexible drug cord may comprise rotating pinch rollers to draw the length of flexible drug cord from the spool and advance that length to a port on the enclosure to the target tissue site. Alternatively, unwinding the spool to advance the length of the flexible drug cord may comprise engaging the drug cord length with a reciprocating linear driver to draw the length of the flexible drug cord from the spool and advance the length to a port on the enclosure to the target tissue site. In still other cases, when the drug cord has sufficient column strength, the spool can be driven to advance a length of the drug cord from the housing or enclosure.

In many embodiments, the length of the flexible drug cord which is delivered from the enclosure will be advanced into a diffusion chamber to expose the length of the drug cord to body fluids (e.g., blood, CSF, etc.) and dissolve the length of the drug cord to form a solution comprising the drug which diffuses out of the diffusion chamber to the target tissue site. Usually, the diffusion chamber is attached or otherwise coupled to the enclosure by a catheter or other tubular connector so that the target tissue site can be treated while the enclosure is implanted remotely from the target tissue site.

Embodiments of the methods of the invention using a drug cord, typically further comprise cutting the flexible drug cord at a predetermined length to dispense said length of drug cord which will degrade as a single dose to be delivered to the patient. The extended period for maintaining the drug in the patient may last for up to five years. The flexible drug cord may be delivered at regular intervals, such as from one hour to seven days, from one hour to two days, from one hour to one day, from one hour to twelve hours, and from one hour to two hours. Alternatively, the flexible drug cord length may be delivered in response to a sensed physiologic parameter, such as a neurological seizure, an epileptic seizure, and erythema, hypertension, hyperglycemia, or the like. Alternative drugs can be provided for the treatment of epilepsy, the treatment of arrhythmia, the treatment of diabetes, the treatment of angina, and the like.

Further details of these and other embodiments and aspects of the invention are described more fully below, with reference to the attached drawing figures.

### BRIEF DESCRIPTION OF THE DRAWINGS

FIG. 1 illustrates a first exemplary embodiment of a solid drug dispensing apparatus constructed in accordance with the principles of the present invention having opposed pinch rollers advancing a drug cord to a drug treatment site.

FIG. 2 illustrates an alternative drug advancement mechanism comprising a linear actuator which may be used in the embodiment of FIG. 1.

FIGS. 2A-2J illustrate various embodiment of the drug cord; FIGS. 2A and 2B illustrate embodiments of the cord without (2A) and with a stiffening core (2B) and an overlying drug layer; FIGS. 2C and 2D illustrate embodiments of the drug cord having a notch or other feature for separating the cords into preset lengths and doses; FIGS. 2E-2G illustrate embodiments of the drug cord having a grooved surface for enhancing dissolution of the cord in tissue fluids; FIG. 2H, shows an embodiment of the drug cord having a helical shaped groove pattern; and FIGS. 2I-2J illustrate shows use of helical or other curved groove pattern to induce or create a vortex flow around the drug cord.

FIG. 3 illustrates an embodiment of an exemplary manner of attaching a catheter to an enclosure for use in the apparatus of the present invention.

FIGS. 4A-4C illustrate an exemplary protocol for advancing, cutting, and dissolving lengths of a drug cord in order to release dissolved drug to a target treatment site in accordance with the principals of the present invention.

FIGS. 5A-5G illustrate various cross section profiles of the drug cord, including round, FIG. 5A; oval FIG. 5b; rectangular, FIG. 5c; triangular FIG. 5d; diamond, FIG. 5e, curved top and flat/rectangular bottom, FIG. 5f; and curved top and pointed bottom, FIG. 5g.

### DETAILED DESCRIPTION OF THE INVENTION

Embodiments of the invention provide apparatus, systems, formulations and methods for delivering medications in solid form to various locations in the body. Many embodiments provide an implanted apparatus for delivering medication in solid form wherein the solid medication form medication comprises a string or cord and includes one or more solid form drugs or other therapeutic agents for treating various medical conditions such as epilepsy, cancer such as glioblastoma and other brain cancers, diabetes, high blood pressure, and high cholesterol. Particular embodiments provide an implanted apparatus for delivering solid form medications in the form of a drug cord or string which is advanced into a diffusion chamber where it dissolves in tissues fluids to diffuse out of the chamber to be delivered to a target tissue site TS (herein target site TS), such as the heart, to treat a medical condition for an extended period of time. Embodiments also provide various solid form medications in the form of a string or cord comprising one or more drugs or other therapeutic agents to be delivered by embodiments of the apparatus or other solid drug delivery apparatus. As used herein the term, “about” means within ±10% of a stated property, dimension or other value and, more preferably, within ±5% of the stated value. Also as used herein, the term “substantially” means within ±10% of a stated property or quality (e.g., linearity), more preferably, within ±5% of the stated value.

Particular embodiments of the invention provide novel medicament preparations and implantable drug storage and delivery devices which can dispense doses of medicaments comprising a drug or other form of medicament discussed herein in the form of a long thin flexible cord, string or thread. For ease of discussion, the medicament preparation is herein is described as a “flexible drug cord” or more simply “drug cord”, the other forms described herein are equally applicable. Typically, the drug cord is stored on a spool though other storage means are also considered. According to various embodiments, all or selected portions of the drug cord such the core of the drug cord may have similar mechanical properties to fishing line, in that it is flexible enough to be bent in a tight spool, yet it is stiff and rigid enough to be grasped between compression wheels, a linear advancement mechanism, or a similar mechanism so as to be pushed from an enclosure and optionally through a lumen of a catheter or other tubular device to a selected drug delivery site.

In particular embodiments, the drug cord may be stored in a tightly wound coil onto a spool in the drug storage device to minimize storage volume. This drug dispensing device typically has an electro-mechanical drive system for drawing selected lengths of the drug cord from the spool and advancing, e.g. pushing, the drug cord through a port on the enclosure and optionally catheter lumen to deliver the drug cord to the tip of this catheter. The port on the enclosure and/or lumen of the catheter will have one or more seals to minimize moisture intrusion into the enclosure, particularly to the volume within the enclosure where the drug cord is stored on the spool.

In specific embodiments, the drug cord exits the enclosure and the catheter lumen into a diffusion chamber at the catheter tip. Body fluids can flow into the diffusion chamber to dissolve/degrade the exposed drug cord to solubilize and release the drug stored in the drug cord. Drug release could occur continuously, periodically on a predefined schedule, based on manual command, and/or based on sensed physiological condition(s). For embodiments employing automatic control, the implantable device can contain or be connected to implanted or external physiological sensor(s), and the sensors can be coupled to a microcontroller configured to assess sensed physiologic condition(s) and to initiate and control dispensing of the drug cord to deliver the drug.

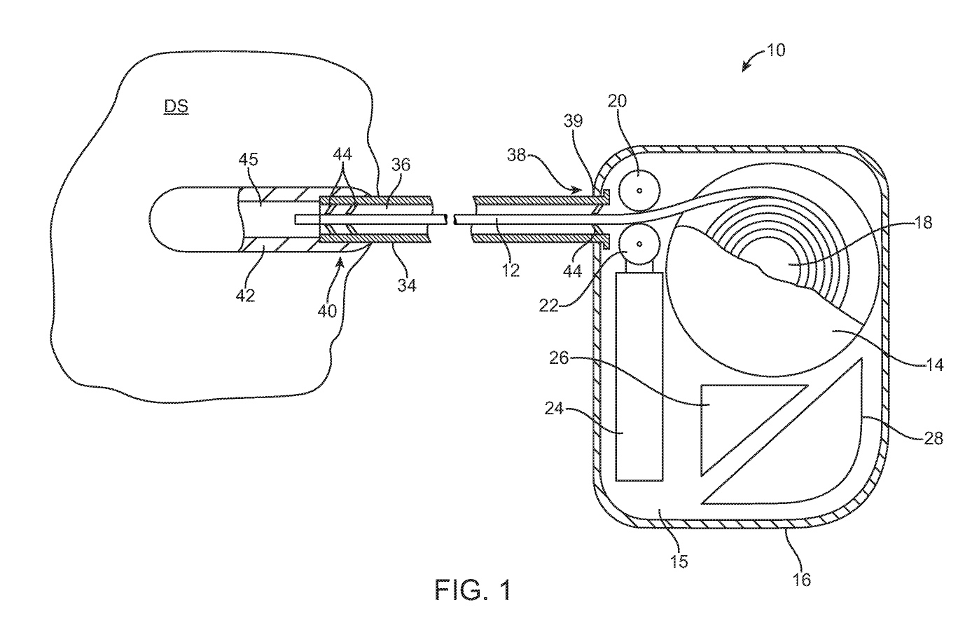

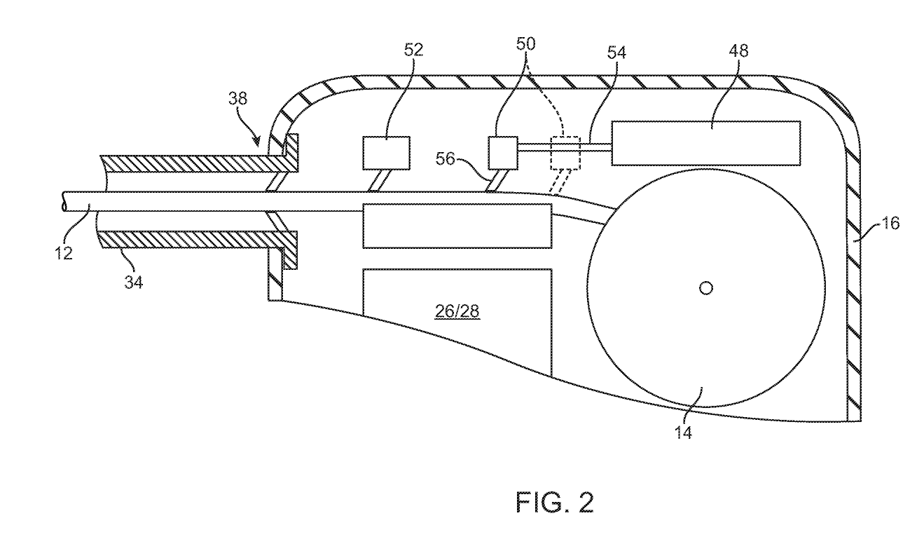

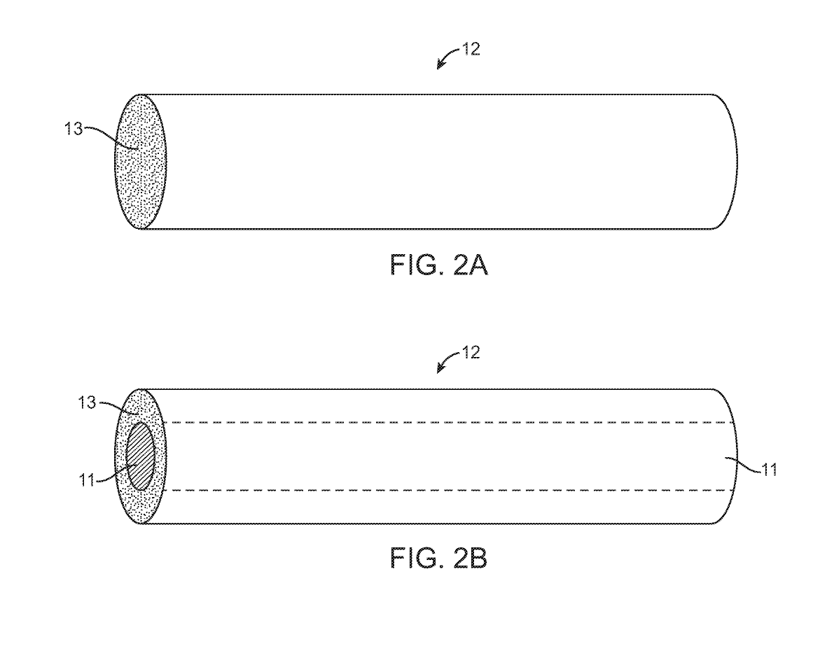

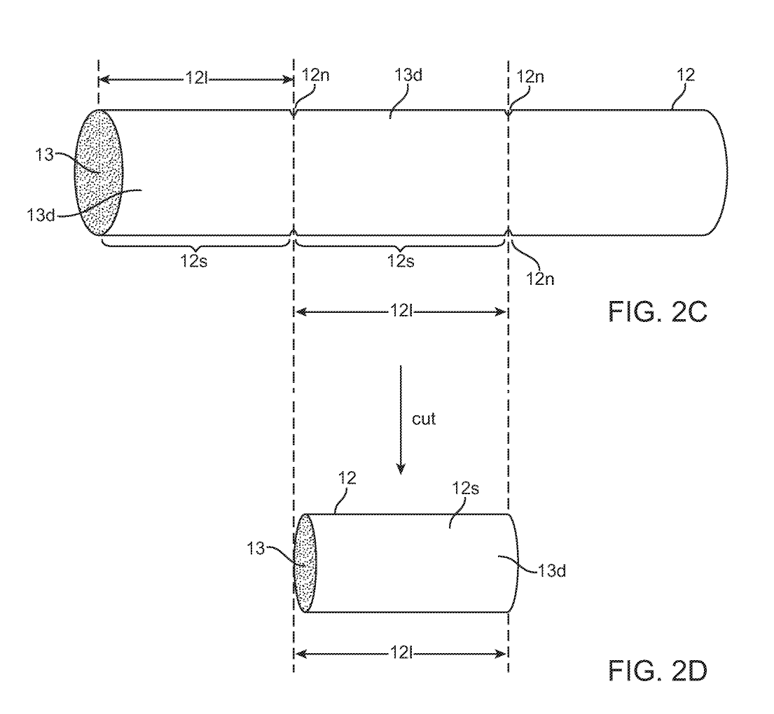

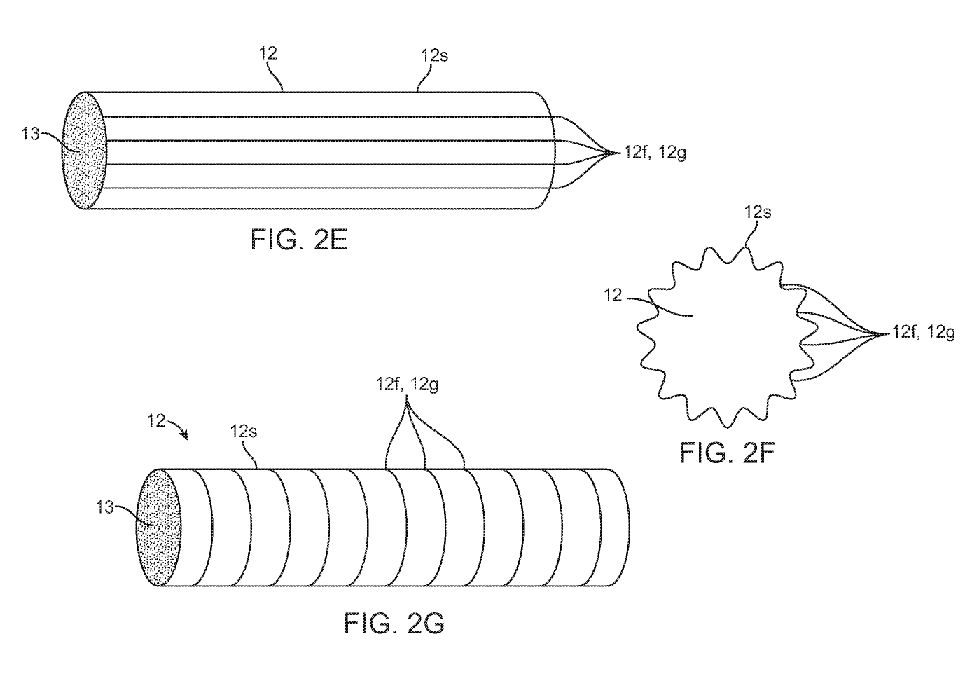

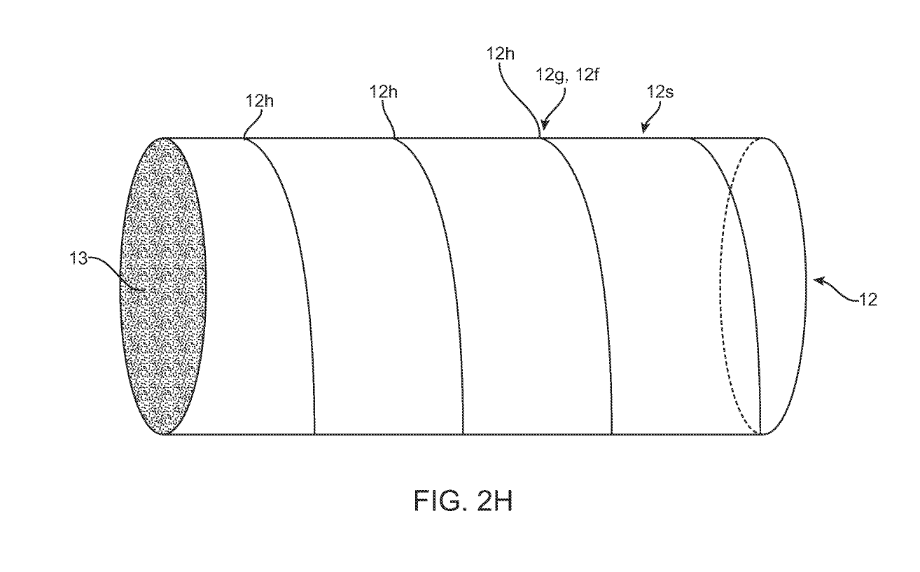

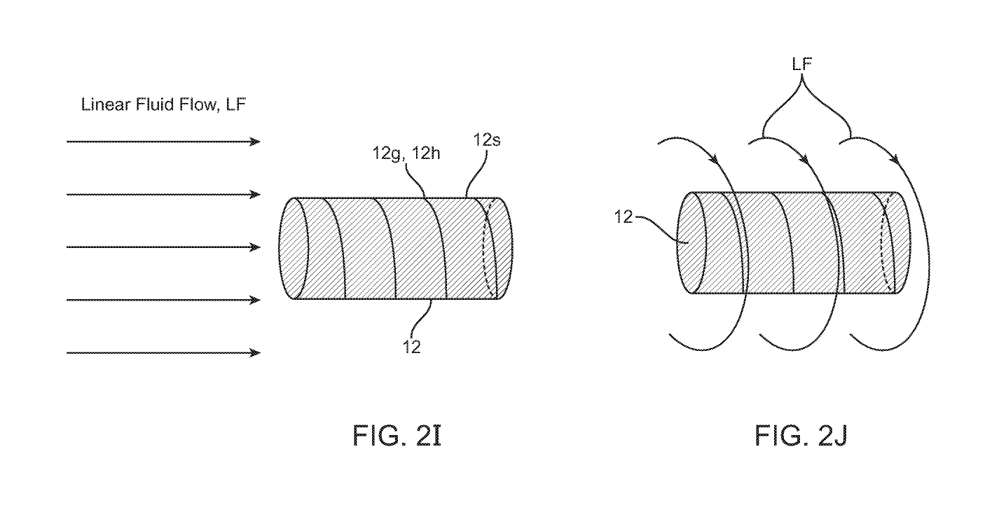

Referring now to FIGS. 1 and 2A-2B, an embodiment of a medical drug delivery apparatus 10 constructed in accordance with the principles of the present invention is configured to deliver a flexible drug cord 12 comprising a medicament 13 from a rotatable spool 14 disposed within an interior 15 of an enclosure 16. It should be appreciated that the drug cord can be stored on other storage devices, rotatable and non rotatable, besides spool 14. The flexible drug cord 12 is wound over a spool core 18 so that the drug cord is stored in a compact, layered structure in a manner similar to fishing line on a fishing line spool.

A discussion will be will now be presented of the flexible drug cord 12. According to various embodiments, the drug cord will comprise a matrix material 12m containing a medicament 13 which is dispersed in the matrix material. The matrix material is degradable in a physiologic environment (e.g. such as in the CSF of the brain or blood in the heart) and desirably non-degradable in a hermetically sealed environment. The matrix material 12m may comprise various biodegradable polymers known in the art, a preferred example include polyethylene oxide (PEO) for example PEO 100K or PEO 200k available from the Dow Chemical Corporation. Other examples may include various poly-lactic acid (PLA) and poly(lactic-co-glycolic acid) (PGLA) as well as various hydrogels known in the art. At least one drug will be disbursed or otherwise distributed within at least a portion of a length of the matrix material. Desirably the medicament 13 is uniformly dispersed in the matrix material 12m, however non uniform dispersions are also considered such as drug dispersed only in the core of the cord or on a surface layer. Radial gradient dispersions are also contemplated with embodiments including higher concentrations of drug in the center of the cord and vice versa. The matrix material formed with the at least one drug is desirably configured to be (i) wound and stored on a spool in a housing implanted within the patient's prosperous body for an extended period without substantial degradation or deleterious effect to the medication, (ii) delivered to a delivery site by unwinding from the spool and advancing through a port in the housing, and (iii) dissolved in tissue fluids at a target tissue site to release the drug and produce a therapeutic affect at the target tissue site to treat a disease or condition. The formation of the drug cord including the drug may accomplished by a variety of means known in the polymer and pharmaceutical manufacturing arts such as extrusion (see the Examples for further description of particular extrusion processes), molding etc. In particular embodiments the matrix material 12m may be melted and then the medicament added (either in solid or liquid form) and mixed to achieve a uniform dispersion of medicament within the matrix material prior to cord formation, e.g., by extrusion, molding etc.

The flexible drug cord 12 will typically be drawn from the spool 14 by a driver, for example, comprising a pair of opposed pinch rollers 20 and 22 where roller 22 is driven by motor 24 and idler roller 20 is undriven. The motor 24 will be controlled by a control unit 26 also located within the housing. The control unit 26 will control the motor to dispense a preselected length of the flexible drug cord 12, where the length can be determined by any of the techniques described previously. Both the controller 26 and motor 24 are powered by a battery 28 or other power storage device which may be rechargeable as is common for implantable medical devices. While the driver will most often be configured to draw or pull the flexible drug cord 12 from the spool 14, in some cases where the flexible drug cord 12 has sufficient column strength, the cord can be advanced by rotating the spool with a stepper motor or other servo-controlled motor (not illustrated). In various embodiments, the column strength of the drug cord can range from about 0.1 to 10 lbs, with specific embodiments of 0.2, 0.25 0.5 1, 1.5, 2, 3, 4, 5, 6, 7, 8 and 9 lbs. Table 1 below illustrates the buckling force for selected lengths and diameters of the drug cord assuming a modulus of elasticity of 920 Mpa which was the average Youngs Modulus for samples of drug cord made from PEO and Taptoec as described in Example 1. The buckling force was calculated using the outlined equation. Desirably the buckling force should not be less than about 1 Newton or 0.225 pound force, which indicates that the drug cord length should not be more than about 10 mm for drug cord diameter of 0.6 mm; no more than 16 mm for a diameter of 0.75 mm and no more than 24 mm for a drug cord diameter of 0.9 mm. Other buckling force results are shown in Example 2 for Furosemide.

F=nπ² E I/L² F force, Newtons
n column condition, here n=2
E modulus of elasticity, Mpa=920
I second moment of area, mm⁴
L is drug cord length, mm
I=π d⁴/64
d is drug cord diameter, mm

TABLE 1
Buckling force data for PEO + Topotecan (1%)

| | 0.6 mm | 0.75 mm | 0.9 mm |
|---|---|---|---|
| L\d | d = 0.6 mm | d = 0.75 mm | d = 0.90 mm |
| 4 | 7.22 | 17.63 | 36.55 |
| 6 | 3.21 | 7.83 | 16.25 |
| 8 | 1.81 | 4.41 | 9.14 |
| 10 | 1.16 | 2.82 | 5.85 |
| 12 | 0.80 | 1.96 | 4.06 |
| 14 | 0.59 | 1.44 | 2.98 |
| 16 | 0.45 | 1.10 | 2.28 |
| 18 | 0.36 | 0.87 | 1.81 |
| 20 | 0.29 | 0.71 | 1.46 |
| 22 | 0.24 | 0.58 | 1.21 |
| 24 | 0.20 | 0.49 | 1.02 |
| 26 | 0.17 | 0.42 | 0.87 |

In addition to bucking forces bending radius were also determined for various drug cords samples obtained in Examples 1 and 2. The bending radius of the drug cord is equal to the radius of the sample multiplied by Youngs' Modulus divided by bending stress (assume a bending stress and tensile stress ware nominally the same). Assuming a tensile stress, bending radius of numerous radiuses of drug cord were calculated using the equation below. The results are summarized in tables 6-8 and 10 As the bending radius decreases, the tensile stress increases. Bending radius data provides the radius that a given sample of drug cord may be bent to with a specific amount of force and may used to predict what spool diameter of spool core 18 a given sample of drug cord may be wrapped around

$$R = \frac{r}{σ} E$$

Where R=bending radius, r=sample radius, E=Young's modulus and σ is bending stress.

Various embodiments of the drug cord for achieving such column strength can include one or more of the following: i) the use of crosslinking of the molecular components comprising drug cord 12, ii) the use of stiffening agents in the drug cord 12, and/or iii) the use of a stiffening core 11 placed within the drug cord 12. Typically, core 11 will be a polymer core comprised of biodegradable materials known in the art and may or may not contain drug or other medicament 13. Suitable biodegradable materials include PGLA, PGA, PEO the other materials area also considered. The stiffening polymer core may be positioned within the drug cord 12 by one or more of insertion, co-extrusion (e.g where a degradable layer comprising drug is coextruded over the core, or dip coating of the core.

In addition to column strength other properties of the drug cord may be selected to have it be sufficiently flexible to wound on the spool as well track through a catheter 34. Described herein

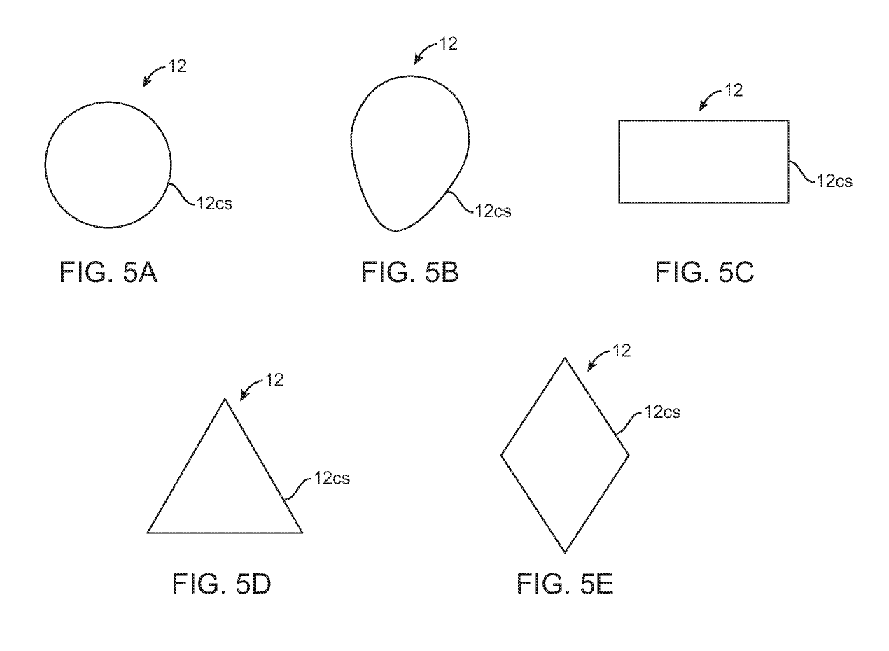

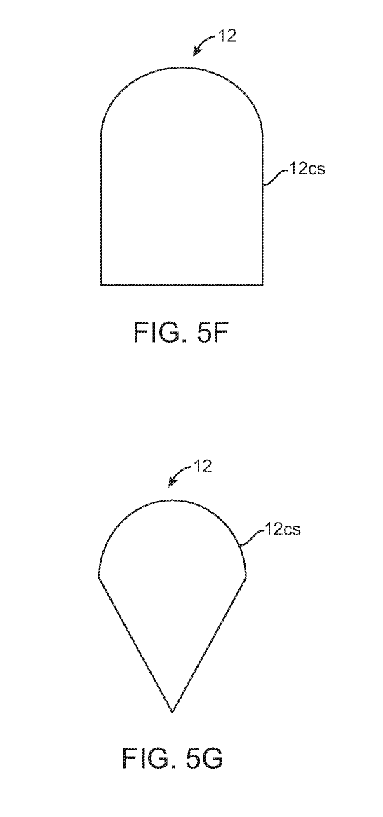

Referring now to FIGS. 5a-5g, various shapes are contemplated for drug cord 12 including the shape of its cross section or profile 12cs. Typically, the drug cord 12 will have a round cross section as shown in FIG. 5a. However, in alternative embodiments the drug cord may have one or more of an oval (FIG. 5b), rectangular (FIG. 5c), triangular (FIG. 5d) or diamond cross (FIG. 5e) section as shown in FIGS. 5b-5e. In still other embodiments, it may have a hybrid profile, for example round on one side and square (FIG. 5f) or pointed (FIG. 5g) on the other side as shown in FIGS. 5f and 5g. The particular cross section may be matched to the interior surface of spool core 18 or other portion or element of apparatus 10

As described herein, selected lengths of drug cord 12 can be cut by a cutting means such as a cutter sleeve 90 which is included with or otherwise associated with apparatus 10. Referring now to FIGS. 2C-2D, in other embodiments, the drug cord 12 may be partially pre-cut (or otherwise scored, notched or shaped or treated to facilitate separation) at set lengths corresponding to a desired dosage of drug. For partially pre-cut embodiments, the separation of drug cord 12 can be effected by still other means, such as applying force in the form of tension, bending/shear, torsional or other like force. Also, in or more embodiments, the drug cord 12 may have sections 12s repeated at regular intervals that include a notched, scored or other separation feature 12n configured to facilitate the cutting (or other form of detachment) and dispensing of fixed lengths 121 of drug cord 12 from a spooled length of drug cord on spool core 18. According to one or more embodiments, the fixed lengths 121 of drug cord 12 may correspond to set dosages 13d of a given drug or other medicament 13 as is described below. In various embodiments, the set dosages may correspond to amounts of a drug from about 0.1 to 50 mgs, with a narrow range of 0.5 to 10 mgs and particular embodiments of 1, 2, 3, 4, 5, 6, 7, 9 and 10 mgs.

In various embodiments, the physical, material and chemical properties of the drug cord 12 and/or its material composition can be configured to achieve a desired rate of the drug dissolution. In particular embodiments selected dissolution rates can be achieved through the selection of the excipients 12e used to fabricated into the drug cord. In other embodiments, selected dissolution rates can be achieve through the use of features incorporated into the drug cord. Referring now to FIGS. 2E-2J, in various embodiments, drug cord 12 can include disintegration and/or dissolution enchaning features 12f configured to enhance the rate of dissolution of drug cord 12 in various bodily fluids such as blood, CSF, interstitial fluid, etc. Dissolution features 12f may correspond to one or more of grooves (including helical grooves), pores or convolutions on or within the surface 12s of drug cord 12. Among other effects, features 12f serve to enhance the surface area of drug cord 12 with tissue fluids and thus, the rate of dissolution of the drug cord by providing more surface area for contact with bodily fluids such as CSF, blood, etc. In particular embodiments, dissolution features 12f correspond to grooves including linear groove (as showing in the embodiment of FIGS. 2E and 2F and curved grooves (as shown in the embodiment of FIG. 2G) including helical grooves (shown in the embodiment of FIG. 2H) which are configured to channel the flow of tissue fluid over cord surface 12s and thus breakdown any stagnant boundary layers that may slow down the diffusion of drug or other therapeutic agent from cord surface 12s. Grooves 12g may also be configured to actually induce flows in different directions over surface 12s further enhancing dissolution. For example, in the embodiments depicted in FIGS. 2H-J, grooves 12g can be positioned around the perimeter of the drug cord in a helical pattern 12h so as to induce radial or vortex flow VF v around the perimeter of the drug cord in response to linear flow LF along an longitudinal length of the drug cord 12. The vortex flow VF serves break to down boundary layers around the drug cord and also creators and/or increases the velocity of fluid moving over drug cord, which in turn increase the rate of mass transfer from the surface of the drug cord by convention. The particular helical or other groove pattern 12h can be matched to the delivery site and/or the biological fluid (including flow rate) at that site, for example the CSF in the brain, CSF in the spinal column, blood in the heart, blood in an arterial site, blood in a venous site, or GI fluid at GI site such as the stomach, small intestine or pancreas.

As described above, drug cord 12 comprises as at least one medicament 13. As used herein, the term “medicament,” refers to any drug, active substance, biological substance, polypeptide, small molecule, protein, or antibody or the like that can be beneficially administered to the patient at a tissue target site whether for therapeutic or diagnostic purposes. Typically, medicament 13 will be referred to as a drug or therapeutic agent 13, though other terms described herein are equally applicable. Suitable medicaments 13 may include a drug for the treatment of epilepsy, such as furosemide; a drug for the treatment of an arrhythmia such as atropine, lidocaine or flecainide; a drug for the treatment of diabetes or other glucose regulation disorder; and a drug for the treatment of angina such as nitroglycerine; or a chemotherapeutic agent or other drug for the treatment of cancer (including glioblastoma or other brain cancer) such as Topotecan. Also medicament 13 may comprise multiple medicaments contained in a single drug cord 12. Tissue target sites TS will typically be in solid tissue, such as muscular tissue, adipose tissue cerebral tissue, solid organ tissue, or the like. Target tissue may also include the patients' blood stream as well as the CSF. Optionally, in addition to the matrix material, the drug cord 12 may comprise a pharmaceutical excipient 12e intended to interact with the medicament in a synergistic or other manner to produce a desired effect on the medicament, its delivery or release in the body. According to these and related embodiments, such excipients 12e may include, for example, disintegrants, preservatives, bulking agents, stiffening agents, cross linking agents and other excipients known in the art. In particular embodiments, the drug cord may include super-disintegrants so as to achieve rapid dissolution of the drug cord in bodily fluid such as blood, CSF or interstitial fluid. In various embodiments, the dissolution rates of the drug cord can range from about 0.01 mg/minute to about 1000 mg/minute, with specific embodiments of 0.02, 0.04, 0.2, 0.4 1, 5, 10, 20, 50, 100, 250, 500, 750, 800 and 900 mg/minute In specific embodiments of the drug cord 12 which include Topotecan, the dissolution rates can be in the range of about 0.01 to about 0.04 mg/hour

The flexible drug cord 12 will pass through at least one seal 44 as it exits from the enclosure 16. According to many embodiments, seal 44 is a flexible seal such that it bends or flexes when drug cord 12 passes through it. The shape of the seal 44 can be matched to the particular cross section 12c or other feature of the drug cord 12. While in some cases the flexible drug cord 12 may be delivered directly to a target tissue site exterior to the site of implantation of the enclosure 16, more usually, the flexible drug cord will pass into a diffusion chamber 36 attached at a distal end 35 of catheter 34 which is attached to a port 39 through a wall of the housing. In particular, the proximal end 38 of the catheter 34 will typically be sealed to the port 39 (e.g, by an adhesive or heat seal), and at least one lumenal seal 44 will be located in the proximal end of the lumen 36 of the catheter 34.

In some embodiments, the flexible drug cord 12 will emerge from the distal end 35 of the catheter 34 and be released directly into the tissue site. Catheter lumen 36 will typically include at least one seal 44 to prevent or inhibit intrusion of body fluids and other materials into the catheter lumen and eventually back into the interior of the enclosure. In prepared embodiments catheter lumen 36 includes two or more seals 44 may be provided at the distal end 37 of the catheter lumen 36 in order to prevent or inhibit intrusion of body fluids and other materials into the catheter lumen and eventually back into the interior of the enclosure 16. Usually, however, the diffusion chamber 42 will be attached at the distal end 35 of the catheter 34 in order to receive an exposed end of the flexible drug cord as it is advanced past the distal end 35 of the catheter 34 and into an interior 45 of the chamber. The diffusion chamber 42 will have a permeable or perforated wall that allows the inflow and exit of body fluids into the interior of the chamber, thus exposing the end of the drug cord 12 to the body fluid, allowing an exposed length of the drug cord to dissolve into the body fluids within the chamber. Once dissolved, the drug or other medicament will be able to diffuse out of the diffusion chamber 42 whose walls will be made of a permeable or perforated material having a pore or perforation size sufficient to allow the diffusion and release of the drug therethrough.

The catheter 34 may comprise any one of various known medical tubing structures having an inner lumen 36 sized to allow advancement of the flexible drug cord 12 therethrough. In various embodiments, the diameter of inner lumen 36 can range from about 0.001 to about 0.3 inches with specific embodiments of 0.005, 0.01, 0.05, 0.1 and 0.2 inches. Catheter 34 typically has a proximal end 38 which is coupled to a port or other opening 39 in the enclosure 16 and a distal end 35 which is coupled to diffusion chamber 42. Catheter 34 typically has a length sufficient to allow positioning of the diffusion chamber 42 in or near a selected tissue delivery or target site DS to allow for diffusion of drug from the diffusion chamber 42 to the delivery site. Catheter 34 can be fabricated from various elastomeric or other flexible polymers such that it be placed in curved or bent position with minimal amount of force and without constriction of the lumen 36. Suitable materials for 34 catheter may include one or more of urethane, silicone, PEBAX, Polyethylene (PE), high density polyethylene (HDPE), polytetrafluoroethylene (TEFLON) and copolymers therefore. The body of the catheter may contain a braid, coil or other structure to reinforce the inner lumen 36 or other inner lumen not shown. Portions or all of the inner lumen 36 may also be coated with a lubricious material and/lined with a coil wire to facilitate advancement of the flexible drug cord there through. Suitable materials for the coil wire including nickel cobalt alloys such as MP35N available from Fort Wayne Metals.

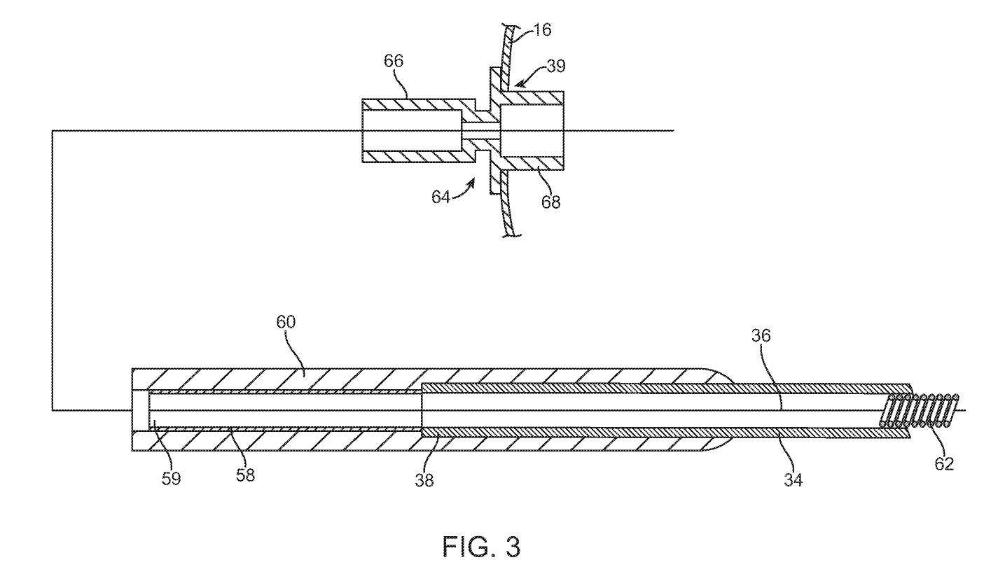

Referring now to FIG. 2, according to another embodiment, instead of the use of pinch rollers (such as pinch rollers 20 and 22 of the embodiment of FIG. 1), the flexible drug cord 12 may be advanced by a linear actuator 48 having a reciprocating engagement member or “gripper” 50 and a stationary engagement member or gripper 52. The linear actuator 48 typically comprises a motor, solenoid, ultrasonic driver, or other mechanism which drives a threaded or other rod 54 which reciprocates the engagement member 50 between the position shown in full line and broken line in FIG. 3, so that an engagement element 56 on the member 50 can grip and advance the drug cord 12 in a forward direction but not in a reverse direction. The stationary gripper 52 helps prevent the flexible drug cord 12 from being drawn in the reverse direction (i.e., toward the right in FIG. 3) by the reciprocating engagement member or “gripper” 50.

Referring now to FIG. 3, an exemplary embodiment of an assembly for coupling the proximal end 38 of the catheter 34 to the port 39 in the enclosure 16 will be described. The assembly comprises an inner connector tube 58 which is covered by an outer elastic sleeve 60, typically a silicone sleeve. The outer sleeve 60 bridges over the inner connector tube 58 and the proximal end 38 of the catheter 34 to provide a hermetic seal. An opposite end 59 of the inner connector tube 58 is received in an outer male port 66 of a fitting 64 which in return is received in the port 39. The silicone or other elastic sleeve 60 is received over the outer surface of the male port 66. The fitting 64 also includes an inner male port 68 which receives the flexible cord 12 as it passes outside of the enclosure. Optionally, all or a portion of the catheter lumen 36 and a lumen of the inner connector tube 58 can be lined with a coil 62 to enhance hoop strength and facilitate passage of the flexible drug cord there through.

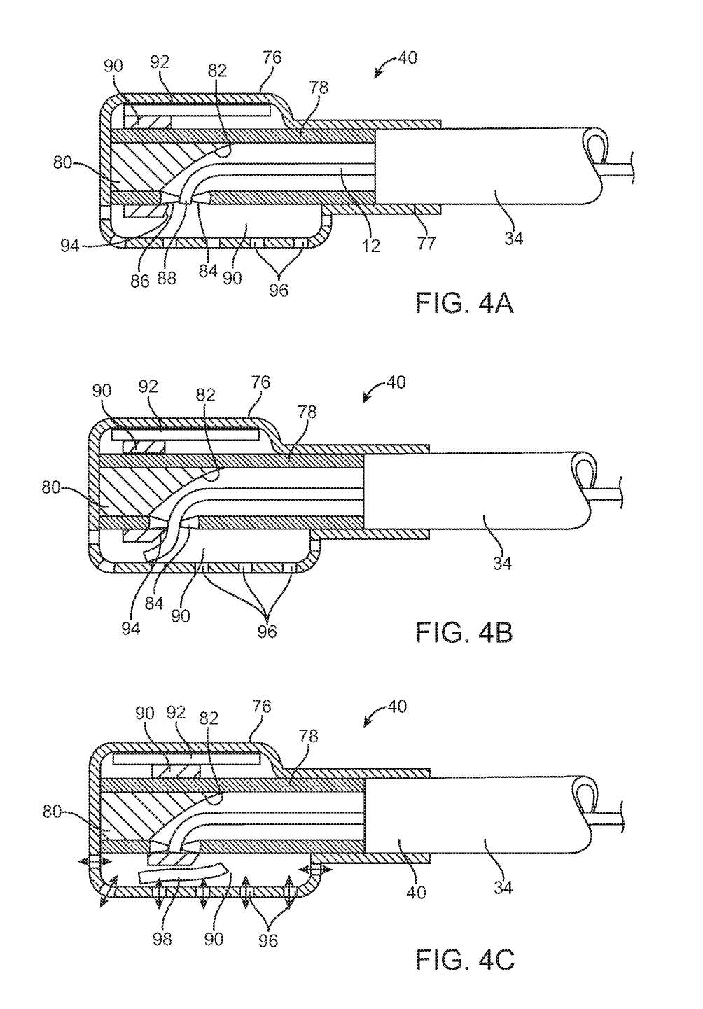

Referring now to FIGS. 4A-4C, an exemplary embodiment of a diffusion chamber 74 having a cutting mechanism 70 for cutting length of the drug cord 12 will now be described. The diffusion chamber 74 comprises an enclosure 76 having an inner tubular guide 78. A shank portion 77 of the enclosure 76 is received over the distal end 35 of the catheter 34 with the proximal end of the inner tubular guide 78 abutting the distal tip of the catheter. An exit guide 80 having a shaped guiding surface 82 is disposed at the proximal end of an inner lumen of the tubular guide 78. The shaped guiding surface 82 acts to deflect a distal tip 88 of the flexible drug cord 12 toward a port 84 in the tubular guide 78. Thus, as the flexible drug cord 12 is advanced, it will enter into an interior volume 75 of the diffusion chamber which defines the diffusion chamber where the drug is dissolved in a body fluid. The distal tip 88 of the drug cord 12 passes through an annular seal 86 disposed in the port 84 in the tubular guide 78. As the flexible drug cord 12 is advanced, the tip 88 is deflected by the shaped guide surface 82 of the exit guide 80 in order to enter the port 84.

A cutter sleeve 90 is co-axially mounted over the inner tubular guide 78 and may be advanced and retracted by a driver 92 disposed within the diffusion chamber 74. As shown in FIG. 4A, the distal tip 88 of the drug cord 12 has not yet been advanced into the chamber portion 90 where dissolution occurs. As shown in FIG. 4B, the drug cord 12 is advanced so that a distal portion 12d thereof advances into the chamber 74. Once a sufficient portion of drug cord has been advanced, the external cutter sleeve 90 is advanced as shown in FIGS. 4B and 4C in order to cut a length 121 of drug cord 12 which is released into the diffusion chamber 74 for dissolution in one or more tissue fluids.

Exemplary seals 44 may comprise a resealable septum allowing a length of drug cord 12 to be passed through the septum with minimal or no intrusion of fluids into the enclosure 16, as is shown in the embodiments of FIG. 1. Such seals 44 may comprise various elastomeric polymers, such as silicone or polyurethane, which have sufficient resilience to open and then seal over the exterior of the drug cord 12 as it is advanced. In particular embodiments, seal 44 may include a re-sealable slit that is normally in a closed and is opened by the passage of the drug cord. In some embodiments, multiple seals may be used and/or individual seals may have multiple septums.

Enclosure or housing 16 can correspond in size to containers used for various pacemakers, with larger and smaller sizes contemplated depending upon for example, the size and configuration of components within the housing. The housing may be fabricated from various biocompatible metals and plastics known in the art, for example, PET, fluoropolymer, PEBAX, polyurethane, titanium, stainless steel and the like. Also, the interior surface or exterior surface of the housing may coated with gas/water vapor impermeable materials or include gas impermeable layers so as to minimize the transmission of water vapor into housing interior. Suitable gas/water impermeable materials include isobutyl rubbers. Enclosure or housing 16 can also include one or more biocompatible coatings known in the art including polyurethanes, silicones, fluoropolymers, PARYLENE, DACRON®, and the like. Coatings can also include various eluting drugs such as various steroids known in the cardiovascular implant arts for reducing the amount of cellular and other bio-adhesion to the housing. Housing 16 can be sized and shaped to fit in various locations in the body including: the skull and cranial cavity, the chest, within in one or more GI organs, the heart, the vascular system, as well as various subcutaneous and intramuscular locations including the extremities and the trunk. All or portions of housing 20 can also be constructed from conformable materials (e.g., polyurethane silicone and other elastomeric polymers) to conform to the shape of surrounding tissue layers and compartment, e.g., the curvature on the inside of the skull, or the contour of the skin. Conforming materials can also be employed to allow for surrounding body tissue to grow around and reshape the housing during prolonged periods of implantation. In this way, embodiments of the invention having a flexible housing minimize the effect of the housing on the growth and function of surrounding tissue, thus allowing the apparatus to be implanted over very prolonged periods including allowing the apparatus to be implanted in children and remain through adulthood. Various conformable materials can also be used to facilitate implantation of an apparatus according to the present invention using minimally invasive methods. Such materials allow the apparatus including the housing or enclosure 16 to bend, twist or otherwise conform so as to be inserted through surgical ports and guiding devices and then reassume its shape once positioned at the intended implantation site. In particular embodiments, bending and twisting of housing can be further facilitated by the use of flexible joints built in for the housing. Housings can also be sized and shaped to further facilitate implantation using minimally invasive surgical methods. For example, the housing can have a particular size and shape such as a cylindrical shape to enable it to pass through various minimally invasive surgical ports and guiding devices. The housing may also be configured to have a collapsed non-deployed state and an expanded deployed state where the non-deployed state is used for advancing the housing and the deployed state used for once the housing is positioned at a desired location in the body.

In many embodiments, apparatus 10 includes an advancement means 40 configured to advance a selected length of drug cord 12 from spool 14 through catheter 34 and into diffusion chamber 42. According to one embodiment shown in FIG. 1, advancement means 40 may correspond to drive rollers 41 which are coupled to a geared electric motor 42. The drive rollers are configured to slightly pinch the drug cord 12 so as to advance it. In another embodiment shown in FIG. 2, advancement means may comprise a grabbing mechanism 50 and 52 having one or more grabbing elements 56 which are configured to engage the drug cord 12 so as to draw or pull the cord out of spool 14. In these and related embodiments, the grabbing mechanism may be powered by a geared electric motor 48 which is linked to the grabber by a transmission means such as a rod 54, screw or like mechanical element. Other advancement means are also contemplated including, for example, a grabbing based advancement means, a hydraulic based means or a pneumatic based means.

Referring back to FIGS. 1 and 2, in various embodiments, motors 24 and actuators 48 may comprise various examples such as brushless DC motor, stepper motor, or other electro-mechanical drive means. In some embodiments, the motors and actuators may be combined with the addition of various gears or other motion control means (e.g., cams, linkages, etc.) for limiting or reconfiguring the motion. In other embodiments, the motors and actuators may be combined with a synchronizing mechanism or element, such as a belt or chain, to achieve the desired level of synchronization between the motion of the driver and the advancement of the cord 12. In still other embodiments, the motion of motor or actuator can be synchronized electronically using controller 26, for example, using one or more software modules as known in the electrical-based drive means art.

Also in particular embodiments, catheter 34 can be configured to provide all or a portion of the driving force for advancing a drug cord 12 from housing 16 to delivery site DS. The driving force can comprise a peristaltic like wave of contraction that travels distally along an inside length of the catheter which acts to grip and advance the drug cord 12. This can be achieved by constructing catheter 34 from either a piezoelectric or like material and coupling it to a voltage source or a shape memory material and coupling it to a thermal power source as is described herein. In the former case, the application of a voltage causes contraction of the catheter material and in the latter case, the application of heat does so. In an alternative embodiment for transporting a length of drug cord 12 through catheter 34, the drug cord can be charged or include a charged coating, such that the drug cord is repelled from the catheter by the application of an electric voltage (having an opposite charge) to the catheter surface.

Desirably, distal catheter tip 35, if used without a diffusion chamber, will have an atraumatic configuration to allow for extended periods of implantation at the target delivery site DS. This can be achieved by configuring the tip to have a tapered shape as well as fabricating the tip from one or more atraumatic flexible polymeric materials including, for example, silicones polyurethanes, fluoropolymers, hydrogels, polyether block amides (PEBA) and others known in the art. Examples of specific atraumatic materials include silver-hydrogel and PEBAX®, a form of PEBA. Catheter 34 including distal tip 63 can also include one or more sensors 64 for making various measurements at the delivery site DS. Such measurements can include one or more of drug concentration, pH, glucose, various metabolites, tissue PO₂ and CO₂ and the like.

According to various embodiments, the apparatus of the present invention can also comprise sensors for making various measurements for determining the degradation/disintegration state of the drug cord 12. Suitable sensors for making such measurements can include optical, impedance, acoustical and chemical sensors and combinations thereof. In various embodiments, the sensors can also be incorporated into an assembly including an emitter and detector. Embodiments of such assemblies can include optical emitters and detectors for making reflectance measurements and ultrasonic transducers (configured as an emitter and detector) for making ultrasonic measurements. Such an assembly sends or emits a signal which is modulated or otherwise altered by the degradation/disintegration state of the cord 12 and then reflected back by cord 12 as a signal which can then be analyzed to determine the degradation state of the drug cord. For example, for use of an optical based assembly, a signal will be returned as a reflected signal which progressively decreases in amplitude as the drug cord is dissolved and disintegrated by body tissue fluids. As indicated above, in various embodiments, cord 12 can include optical or other indicia to facilitate measurement of the degradation state of cord 12.

Embodiments of apparatus 10 having sensors and/or sensor assemblies can be used to control or regulate drug cord delivery by sensing the state of disintegration of previously delivered cords. For example, additional drug cord length can be delivered when it has been determined that the previous release is in a particular state of disintegration (e.g., it has been completely or substantially disintegrated). This determination can be achieved through use of the controller 26 described herein which may include one or more algorithms for analyzing the disintegration state of the drug cord and using this information to make a delivery decision. In particular embodiments, information on the disintegration state of the drug cord can be combined with other data for making a drug cord delivery decision with weightings assignable to each group of data. Such additional data can include for example, the blood/plasma concentration of the delivered drug as well as various physiological data (e.g., temperature, pH, blood gases, etc.) including physiological data indicative of the medical condition to be treated by the delivered drug, e.g., blood glucose as an indication of hyperglycemia, EKG as an indication of arrhythmia or brain electrical activity as an indication of an epileptic seizure or pre seizure event.

In various embodiments, the length of the catheter 34 can be configured to allow the enclosure 16 to be positioned remotely from the delivery site DS. For example, the enclosure 16 can be implanted in the brain with the catheter tip positioned a short distance away (e.g., 0.5 to 5 cms). In another embodiment, the catheter can have sufficient length to allow the distal tip to be positioned in the brain, while enclosure 16 is placed on the scalp or other location outside the skull. In this way, apparatus 10 can be used to deliver medication to a selectable delivery site DS, such as the brain without having to be placed at that site or have any appreciable effect on organs or tissue at that site other than that of the medication itself.

In some embodiments, apparatus 10 can include multiple catheters 34 so as to allow for the delivery of drug cords 12 at multiple locations using a single delivery apparatus 10. For example, the distal tip of a first catheter can be placed at first delivery site and the distal tip of a second catheter can be placed a second delivery site. Alternatively, a first delivery site can comprise the ultimate target site, such as an arthritic joint to allow for immediate delivery of medication to that site and the second catheter distal tip can be placed at a second site at least partially removed from first site such as in muscle tissue or other sub-dermal location to allow for longer term controlled release of a drug.

Apparatus according to embodiments of the present invention will typically include a controller 26 for controlling one or more aspects of the medication delivery process including actuation and control of the drug cord delivery mechanisms. The controller can comprise logic resources such as a microprocessor, a state device or both; and memory resources, such as RAM, DRAM, ROM, etc. Logic resources and/or memory resources may include one or more software modules for operation of the controller. Through the use of modules, the controller may be programmed to include a medication delivery regimen wherein medication is delivered at regular intervals (e.g., once or twice a day, etc.) over an extended period. The controller may also include an RF device for receiving a wireless signal (e.g., wireless or otherwise) to initiate the delivery of medication or to change the delivery regimen (e.g., from once a day to twice a day). In this way, the patient or a medical care provider can control the delivery of medication in response to a specific event (e.g., an episode of angina, an abnormal EKG) or longer term changes in the patient's condition or diagnosis.

The controller 26 can receive inputs from on-board or remote sensor s which senses a physiologic parameter indicative of a condition to be treated by the medication drug cord 12, e.g., diabetic hyperglycemia. When the controller receives an input indicative of the condition, it sends a signal 88 to initiate the delivery of one or more medication cords 12 to the target tissue site so as to treat the medical condition. Both the initial and subsequent inputs from sensor can be used to titrate the delivery of medication cords over an extended period until the condition is dissipated or otherwise treated in a selected manner. The controller can also receive inputs from other sensors which are configured to measure the plasma or other tissue concentration of the delivered drug. These inputs can also be used to titrate the delivery of the drug to achieve a selected concentration of drug. The concentration sensors can be positioned both the target site as well as other sites in the body (e.g., a vein or artery) in order to develop a pharmacokinetic model of the distribution of the drug at multiple sites in the body.

In various method embodiments of the invention, apparatus 10 is used to deliver drug cords 12 to a selected delivery sites, such as subcutaneous tissues, where the cords are disintegrated and absorbed by body tissue fluids (e.g., interstitial fluids in muscle or dermal tissue) so as to produce a desired concentration of drug at a target site. In some embodiments, the delivery site can be in the same organ and/or compartment as the target site, for example the brain. In other embodiments, the target site can be different from the delivery site. For example in one embodiment, the delivery site can be intramuscular tissue in the chest and the target site can be an organ such as the heart which is removed from the delivery site. The delivery site can be oppositional to the target site, for example dermal delivery to reach the target site of underlying muscle tissue, or it can be placed at a non-oppositional site, for example, intramuscular delivery to reach the target site of the heart. In each case, the drug cord 12 can include a selected dose of drug and be configured to disintegrate and be dissolved by body tissue fluids so as to yield a therapeutically effective concentration of the drug at the target tissue site. In many applications, this involves the drug cord being dissolved by body tissue fluids at the delivery site (e.g., interstitial fluids) where the drug then diffuses from the tissue into the blood stream where it is carried to the target tissue site. Accordingly, in these and other applications, the dose of the drug in the drug cord can be titrated to achieve a selected plasma concentration of the drug (or concentration range) for a selected period during and after dissolution of the drug cord.

In some embodiments, drug cord 12 is configured to disintegrate and be dissolved by the tissue fluids within a body compartment such as the cerebrospinal fluid (CSF) in the brain so as to achieve a selected concentration in the tissue fluid within that compartment. In particular embodiments for treating various neural disorders such as epileptic and other seizures, drug cord 12 is configured to rapidly disintegrate and be dissolved in cerebrospinal fluid (CSF) so as to rapidly achieve a selected concentration of the drug throughout the CSF that bathes the brain in order to prevent the occurrence of the seizure or lessen its duration and severity. This can be achieved through the use of one or more super-disintegrants which are compounded into drug cord 12.

In other embodiments, accelerated disintegration of drug cord 12 can also be achieved by treating the drug cord prior to, during or after delivery with mechanical, electromagnetic, acoustical or other energy to weaken the drug cord structure, create cracks for the ingress of fluids or initiate the breakup of the drug cord into smaller pieces. The delivery of force and energy can be used to create cracks (or other surface defects) for the ingress of tissue fluids as well as break the drug cord up into smaller pieces.

Disintegrating energy can be delivered to the drug cord after it is ejected from catheter 34 and delivered to delivery site DS. In such embodiments, energy delivery can be achieved through use of an ultrasonic transducer or other energy delivery device placed on catheter distal tip and/or on the diffusion chamber. Ultrasonic transducer emits a beam of energy which acts upon drug cord 12 to cause cracks and other effects to the drug cord structure to accelerate drug cord degradation into pieces and disintegration through dissolution by body tissue fluids. Other forms of energy which can be used to break up and/or weaken the structure of drug cord 12 and accelerate disintegration/degradation include optical (e.g., laser), RF, microwave, thermal or other forms of energy known in the medical device arts. The energy delivery regimen (e.g., duration, frequency and amount of energy) for weakening the drug cord structure (e.g., causing cracks etc.) can be controlled by controller 26. The energy delivery regimen can be adjusted depending upon the size and structure properties of the drug cord as well as the particular delivery site DS. In various embodiments, energy delivery devices can be powered by power source 28 or have its own power source.

According to the present invention, various medicaments and drug forms are formulated into flexible drug cords 12 by combining, dispersing, or otherwise integrating the medicaments into a biodegradable matrix having properties that allow the cord to be wound over a spool and maintained in an implanted enclosure over extended time periods. Also in many embodiments, the medication cords 12 can be formulated using one or more pharmaceutical excipients. Suitable excipients include preservatives for preserving the drug, binders for binding the drug components together and disintegrants for disintegrating and dissolving the cords in a controlled fashion to achieve and maintain a sufficient concentration of the drug (either at the tissue site or other tissue location) for treatment of the condition. As is described herein, disintegrants can include super-disintegrants known in the art. Example super-disintegrants include, without limitation, sodium starch glycolate, crospovidone, croscarmellose sodium as well as related salts and like compounds.

In various embodiments, drug cords 12 can comprise a single or a plurality of drugs 13. In particular embodiments, drug cords 12 can include a combination of drugs for treatment of a single or multiple conditions, for example, a cocktail of antiviral drugs such as protease inhibitors for treatment of HIV AIDS and also antibiotics for the treatment of adjunct bacterial infections. It may also contain combinations of other antibiotics for treatment of other infections such as septicemia. In other embodiments, the drug cords may contain combinations of chemotherapeutic agents for the treatment of cancer such as topotecan and paclitaxel for the treatment of extensive-stage small-cell lung cancer or other related cancer.

In various applications, embodiments of the invention can be used to deliver drug cords 12 comprising solid form medicaments to provide treatment for a number of medical conditions including for example epileptic seizures (e.g., by use of Furosemide), cancer including brain cancers such as glioblastoma (e.g. by the use of Topotecan); high blood pressure (e.g., by use of calcium channel blockers or CCBs), elevated cholesterol (e.g., by use of statins such as LIPITOR), diabetes (e.g., by use of insulin), coronary arrhythmia's (both atrial and ventricular, e.g., by use of CCB's), coronary ischemia (e.g., by use of nitroglycerin or other vasodilating agent), or cerebral ischemia, heart attack or stroke (e.g., by use of aspirin, TPA or other hemolytic agent), anemia (e.g., by use of ferric-pyrophosphate), hemophilia or other clotting factor deficiency (e.g., by use of factor 8) or other like conditions. Further embodiments of the invention can be used to provide concurrent treatment for two or more of these or other conditions eliminating the need for the patient to take multiple doses of different drugs (e.g., orally or by parenteral means) over the course of a day. This is particularly beneficial to patients who have long term chronic conditions including those who have impaired cognitive or physical abilities.

Embodiments of the Drug Cord Comprising Topotecan

Various embodiments of the invention contemplate a drug cord 12 comprising Topotecan as well as its analogues and derivitives. Further description of such embodiments are described in Example 1. In the example, the Topotecan was formed using polyehtylene oxide (PEO). The PEO used was manufactured by Dow Chem Co. under the tradename Sentry Polyox, with the specifically grade being WSR N10 LEO NF. However, other matrix materials are also contemplated such as PGA or PGLA.

A brief description will now be presented on Topotecan. Topotecan (trade name HYCAMTIN) is a chemotherapeutic agent that is a topoisomerase inhibitor. It is a synthetic, water-soluble analog of the natural chemical compound camptothecin. It is used in the form of its hydrochloride salt to treat ovarian cancer, lung cancer and other cancer types. Topotecan is a semi-synthetic derivative of camptothecin. Camptothecin is a natural product extracted from the bark of the tree Camptotheca acuminata. Other analogues and derivatives of Topotecan include Irinotecan, Camptothecin, Exatecan, Lurtotecan, Belotecan, Rubitecan. Topoisomerase-I is a nuclear enzyme that relieves torsional strain in DNA by opening single strand breaks. Once topoisomerase-I creates a single strand break, the DNA can rotate in front of the advancing replication fork. In physiological environments, topotecan is in equilibrium with its inactive carboxylate form. Topotecan's active lactone form intercalates between DNA bases in the topoisomerase-I cleavage complex. The binding of topotecan in the cleavage complex prevents topoisomerase-I from religating the nicked DNA strand after relieving the strain. This intercalation therefore traps the topoisomerase-I in the cleavage complex bound to the DNA. When the replication-fork collides with the trapped topoisomerase-I, DNA damage occurs. The unbroken DNA strand breaks and mammalian cells cannot efficiently repair these double strand breaks. The accumulation of trapped topoisomerase-I complexes is a known response to apoptotic stimuli. This disruption prevents DNA replication and ultimately leads to cell death. This process leads to breaks in the DNA strand resulting in apoptosis. Administration of topotecan down-regulates its target, topoisomerase-I; therefore, it is dosed to maximize efficacy and minimize related toxicity. Topotecan is often given in combination with Paclitaxel as first line treatment for extensive-stage small-cell lung cancer.

Various embodiments of the drug cord 12 containing Topotecan may be configured to treat glioblastoma or other brain cancer by delivering intracranially drug lengths 121 containing therapeutically effective doses of Topotecan. In various embodiments, such doses may be in the range of 0.01 mg to 0.1 mg with a preferred range of about 0.01 to 0.04 mg. The weight percent of Topotecan in the drug cord to achieve such dosages may be in the range from about 1 to 10%. Further description of a delivery regimen of Topotecan including dosages may be found in the paper by Bruce, J, et al.: Regression of Recurrent Malignant Gliomas With Convection-Enhanced Delivery of Topotecan Neurosurgery 69:1272-1280, 2011, which is incorporated by reference herein for all purposes.

Specific embodiments of the drug cord may have a weight percent of Topotecan's in the range of about 1 to about 20% relative to the total weight of the drug cord, more preferrably about 1 to 10% with specific embodiments of 2, 4, 5, 6, 7, 8, 9 weight %. The amount of Topotecan in a given length of drug cord can be selected to deliver between about 0.01 mg to about 0.04 mg of Topotecan in a selected length of drug cord, for example in the range of about 10 to 25 mm. The youngs modulus of such a drug cord can be in the range of about 860 to 980 Mp, with specific embodiments shown in Table 5 in Example 1. The axial stiffness can be in the range of about 7.9 to 10 N/mm with specific embodiments shown in Table 5. The tensile strength can be in the range of about 11.8 to 14 MPa, with specific embodiments shown in Table 5.

Embodiments of the Drug Cord Comprising Furosemide

Various embodiments of the invention contemplate a drug cord 12 comprising Furosemide as well as its analogues and derivitives such as Bumetanide, Torsemide. Embodiments of drug cord comprising Furosemide may be delivered intra-cranially to treat or prevent epilepsy and other related neurological disorders such as seizures, etc. caused by aberrant electrical signals in the brain. In various embodiments, the weight percent of Furosemide in the drug cord 12 may be in a range from about 1 to 10%, with specific embodiments of 2, 3, 4, 5, 6, 7, 8 and 9%. Further description of such embodiments are described in Example 2. In the example, the Furosemide was formed using polyehtylene oxide (Polyox from Dow Chemical), the specific grade being WSR N10 LEO NF. However, other matrix materials are also contemplated such as PGA or PGLA.

Furosemide is a type of loop diuretic that works by decreasing the reabsorption of sodium by the kidneys and is used to treat heart disease.

### EXAMPLES

Various embodiments of the invention will now be further illustrated with reference to the following examples. However, it will be appreciated that these examples are presented for purposes of illustration and the invention is not to be limited by these specific examples or the details therein.

### Example I

In this example, lengths of drug cord were fabricated using PEO as the matrix material and topotecan hydrochloride (1% by weight) as the medicament. Topotecan hydrochloride is herein referred to as Topotecan. Lengths of cord were prepared with and without Topotecan in order to compare the mechanical and other physical properties of the mixture when the Topotecan was added. The PEO used was SENTRY POLYOX (herein Polyox) obtained from Dow Chemical with the particular grade corresponding to WSR N10 LEO NF. The lot # used was WP389380. WSR N10 refers to the average molecular weight (100 k), LEO stands for low ethylene oxide, and NF indicates that it meets National Formulary requirements. This grade of Polyox is referred to herein as PEO 100k. The Topotecan was obtained from the USP (Cat.#167225, and the Lot# R007C0). In the particular lot made (LOT #510-29-1) 4.95 gram of PEO 100K was combined with 0.05 gram of Topotecan HCl and mixed thoroughly. The mixture was then fed into a twin screw, co-rotating extruder (MiniCTW, Thermo Scientific) set at a barrel temperature of 68° C. and screw speed of 20 rpm. After loading, the molten PEO/Topotecan dispersion was allowed to circulate for 10 min within hopper to allow for drug content uniformity. Extrusion can be controlled either by constant screw speed or, in this case, constant torque. Constant is the torque that is applied to the screws. As the material is extruded out of the barrel, the unit automatically increases the screw speed to maintain the same torque resulting in a uniform thickness of the extrudate throughout the extrusion. The material was extruded under torque control (e.g., constant) torque set at 0.35 Nm for the two PEO 100k lots (with and without Topotecan) and 0.50 Nm for the PEO 200k lot. Three lots of material were made in all, one using just PEO 100k (lot # LOT #510-20-10), one with a higher molecular weight PEO, herein PEO 200k (LOT #510-28-1) and one with PEO 100k and Topotecan (LOT #520-29-1). The PEO 200k was also Polyox obtained from the Dow chemical company and corresponded to grade WSR N80 LEO NF and lot #WP391562. About 16 feet or so of material were made for each lot and the diameters measured at one foot intervals. Table 2 shows the diameters measured for the PEO 100k Topotecan lot

TABLE 2
PEO 100k + Topotecan (1% by weight)

| Mark (ft) | Diameter (mm) |
|---|---|
| 1 | 0.81 |
| 2 | 0.81 |
| 3 | 0.80 |
| 4 | 0.82 |
| 5 | 0.82 |
| 6 | 0.82 |
| 7 | 0.78 |
| 8 | 0.80 |
| 9 | 0.78 |
| 10 | 0.76 |
| 11 | 0.77 |
| 12 | 0.78 |
| 13 | 0.78 |
| 14 | 0.77 |
| 15 | 0.76 |
| 16 | 0.67 |

To better understand the physical characteristics and performance of this PEO/Topotecan drug cord mixture, tensile testing was performed on all three lost. The drug cord specimens were approximately 0.7-0.9 mm in diameter and approximately 80.0 mm in length composed of 4.95 g of PEO and 0.05 g of Topotecan HCl making a tot 5.00 g total. These samples were placed in a tensile tester (Model: Instron 3343) and held in place with miniature grips. The pull test was done in ambient temperature and humidity at the rate of 10 mm/min with a gauge length of 52.0 mm of drug cord. The results which included tensile strength, Young's modulus and axial stiffness of the drug cord samples are summarized below in tables 3, 4, and 5 corresponding to 100% PEO 100K, 100% PEO 200K, and Topotecan 1% HCL in PEO 100k. Bending radiuses and buckling forces were also measured and/or calculated for the various samples. Tables 6, 7 and 8 also provide buckling force and bending radius data for the three lots respectively.

TABLE 3
PEO 100k only

| | | | | Tensile | |
|---|---|---|---|---|---|
| Sample | | Avg. | | Young's | |
| No. | Dia. | Area | Strength | Modulus | Axial |
| (mm) | (mm²) | (MPa) | (MPa) | (N/mm) | Stiffness |
| 1 | 0.77 | 0.47 | 8.63 | 1058.3 | 9.48 |
| 2 | 0.77 | 0.47 | 12.37 | 1063.7 | 9.53 |
| 3 | 0.77 | 0.47 | 13.68 | 1048.2 | 9.39 |
| 4 | 0.77 | 0.47 | 12.95 | 1105.6 | 9.90 |
| 5 | 0.74 | 0.43 | 13.11 | 1028.6 | 8.51 |
| 6 | 0.77 | 0.47 | 11.68 | 1050.6 | 9.41 |
| 7 | 0.77 | 0.47 | 11.36 | 1122.9 | 10.06 |
| 8 | 0.74 | 0.43 | 11.95 | 1028.9 | 8.51 |
| 9 | 0.76 | 0.45 | 10.07 | 1139.1 | 9.94 |

Avg. Tensile Strength (MPa)-11.76
Avg. Young's Modulus (MPa)-1071.79
Avg. Axial Stiffness (N/mm)-9.41
Std. Dev. Tensile Strength (MPa)-1.59
Std. Dev. Young's Modulus (MPa)-40.68
Std. Dev. Axial Stiffness (N/mm)-0.568

TABLE 4
PEO 200k only

| | | | | Tensile | |
|---|---|---|---|---|---|
| | | Avg. | | Young's | Axial |
| Sample | Dia. | Area | Strength | Modulus | Stiffness |
| No. | (mm) | (mm²) | (MPa) | (MPa) | (N/mm) |
| 1 | 0.83 | 0.54 | 10.17 | 663.8 | 6.91 |
| 2 | 0.82 | 0.53 | 10.15 | 664.7 | 6.75 |
| 3 | 0.81 | 0.52 | 10.91 | 634.5 | 6.29 |
| 4 | 0.82 | 0.53 | 10.66 | 642.0 | 6.52 |
| 5 | 0.80 | 0.50 | 9.09 | 552.3 | 5.34 |
| 6 | 0.80 | 0.50 | 10.31 | 641.0 | 6.20 |
| 7 | 0.81 | 0.52 | 9.78 | 654.4 | 6.48 |
| 8 | 0.79 | 0.49 | 9.79 | 623.1 | 5.87 |
| 9 | 0.80 | 0.50 | 9.35 | 680.5 | 6.58 |
| 10 | 0.80 | 0.50 | 9.39 | 614.9 | 5.94 |

Avg. Tensile Strength (MPa)-9.96
Avg. Young's Modulus (MPa)-637.11
Avg. Axial Stiffness (N/mm)-6.29
Avg. Bending Radius (mm)-25.89
Std. Dev. Tensile Strength (MPa)-0.59
Std. Dev. Young's' Modulus (MPa)-35.88
Std. Dev. Axial Stiffness (N/mm)-0.468
Avg. Buckling Force (N)-0.10

TABLE 5
PEO 100k + Topotecan (1% by weight)

| | | | | Tensile | |
|---|---|---|---|---|---|
| | | Avg. | | Young's | Axial |
| Sample | Dia. | Area | Strength | Modulus | Stiffness |
| No. | (mm) | (mm²) | (MPa) | (MPa) | (N/mm) |
| 1 | 0.81 | 0.52 | 13.80 | 976.12 | 9.67 |
| 2 | 0.81 | 0.52 | 12.73 | 907.3 | 8.99 |
| 3 | 0.80 | 0.50 | 11.50 | 898.6 | 8.69 |
| 4 | 0.82 | 0.53 | 13.01 | 922.2 | 9.37 |
| 5 | 0.82 | 0.53 | 11.80 | 931.0 | 9.45 |
| 6 | 0.82 | 0.53 | 12.93 | 990.8 | 10.06 |
| 7 | 0.78 | 0.48 | 13.69 | 863.4 | 7.93 |
| 8 | 0.80 | 0.50 | 13.09 | 900.5 | 8.70 |
| 9 | 0.78 | 0.48 | 13.46 | 899.0 | 8.26 |

Avg. Tensile Strength (MPa)-12.89
Avg. Young's Modulus (MPa)-920.98
Avg. Axial Stiffness (N/mm)-9.01
Avg. Bending Radius (mm)-28.86
Std. Dev. Tensile Strength (MPa)-0.79
Std. Dev. Young's' Modulus (MPa)-40.20
Std. Dev. Axial Stiffness (N/mm)-0.688
Avg. Buckling Force (N)-0.14

TABLE 6
PEO 100k only

| Sample | Est. Bending | Buckling |
|---|---|---|
| No. | Radius (mm) | Force (N) |
| 1 | 47.20 | 0.13 |
| 2 | 33.11 | 0.13 |
| 3 | 29.50 | 0.13 |
| 4 | 32.87 | 0.14 |
| 5 | 29.02 | 0.11 |
| 6 | 34.63 | 0.13 |
| 7 | 38.06 | 0.14 |
| 8 | 31.85 | 0.11 |
| 9 | 42.97 | 0.14 |

TABLE 7
PEO 200k only

| Sample | Est. Bending | Buckling |
|---|---|---|
| No. | Radius (mm) | Force (N) |
| 1 | 27.10 | 0.11 |
| 2 | 26.85 | 0.11 |
| 3 | 23.56 | 0.10 |
| 4 | 24.69 | 0.10 |
| 5 | 24.30 | 0.08 |
| 6 | 24.88 | 0.09 |
| 7 | 27.10 | 0.10 |
| 8 | 25.13 | 0.09 |
| 9 | 29.11 | 0.10 |
| 10 | 26.19 | 0.09 |

TABLE 8
PEO 100k + Topotecan (1% by weight)

| Sample | Est. Bending | Buckling |
|---|---|---|
| No. | Radius | Force |
| 1 | 28.65 | 0.15 |
| 2 | 28.86 | 0.14 |
| 3 | 31.26 | 0.13 |
| 4 | 29.06 | 0.15 |
| 5 | 32.35 | 0.15 |
| 6 | 31.41 | 0.16 |
| 7 | 24.60 | 0.11 |
| 8 | 27.52 | 0.13 |
| 9 | 26.05 | 0.12 |

### Example II

In this example, lengths of drug cord were fabricated using PEO as the matrix material and furosemide (1% by weight) as the medicament. The mixture was prepared using a similar process as that used for the Topotecan. Specifically 4.95 grams of PEO 100k (Polyox WSR N10 LEO NF, Lot# WP389380, 4.95 g) was combined with furosemide (TCI Cat# F0182, Lot# BKTSB, 0.05 g) and mixed thoroughly. The mixture was fed into a twin screw, corotating extruder (MiniCTW, Thermo Scientific) set at a barrel temperature of 68° C. and screw speed of 20 rpm. After loading, the molten PEO/furosemide dispersion was allowed to circulate for 10 min to allow for drug content uniformity. The material was extruded under torque control set at 0.35 Nm to give an extrudate that was 15 ft, 8 in long with following diameters measured in one foot interval with diameters summarized in table 9 below. The lot of material produced was designated as Lot#510-30-1

TABLE 9
PEO + Furosemide (1% by weight)

| Mark (ft) | Diameter (mm) |
|---|---|
| 1 | 0.83 |
| 2 | 0.86 |
| 3 | 0.76 |
| 4 | 0.81 |
| 5 | 0.84 |
| 6 | 0.79 |
| 7 | 0.79 |
| 8 | 0.81 |
| 9 | 0.79 |
| 10 | 0.75 |
| 11 | 0.79 |
| 12 | 0.76 |
| 13 | 0.75 |
| 14 | 0.74 |
| 15 | 0.70 |

Similar mechanical tests were performed as that for PEO 100k and Topotecan lot described above (e.g., use of tensile tester). These measurements were used to measure and/or calculate tensile strength Young's modulus, axial stiffness, bucking force, and bending radius. The results are summarized in Tables 10 and 11 below.

TABLE 10
PEO + Furosemide (1% by weight)

| | | | | Tensile | |
|---|---|---|---|---|---|
| | | Avg. | | Young's | Axial |
| Sample | Dia. | Area | Strength | Modulus | Stiffness |
| No. | (mm) | (mm²) | (MPa) | (MPa) | (N/mm) |
| 1 | 0.82 | 0.53 | 13.63 | 1010.2 | 10.26 |
| 2 | 0.84 | 0.55 | 12.99 | 1003.6 | 10.70 |
| 3 | 0.81 | 0.52 | 13.60 | 957.2 | 9.48 |
| 4 | 0.77 | 0.47 | 13.25 | 834.1 | 7.47 |
| 5 | 0.83 | 0.54 | 12.85 | 1010.4 | 10.51 |
| 6 | 0.81 | 0.52 | 13.78 | 963.0 | 9.54 |
| 7 | 0.81 | 0.52 | 12.44 | 926.5 | 9.18 |
| 8 | 0.81 | 0.52 | 13.56 | 937.8 | 9.29 |
| 9 | 0.82 | 0.53 | 13.35 | 979.5 | 9.95 |

Avg. Tensile Strength (MPa)-13.27
Avg. Young's Modulus (MPa)-958.01
Avg. Axial Stiffness (N/mm)-9.60
Avg. Bending Radius (mm)-29.42
Std. Dev. Tensile Strength (MPa)-0.44
Std. Dev. Young's Modulus (MPa)-55.67
Std. Dev. Axial Stiffness (N/mm)-0.963
Avg. Buckling Force (N)-0.15

TABLE 11
PEO + Furosemide

| Sample | Est. Bending | Buckling |
|---|---|---|
| No. | Radius (mm) | Force (N) |
| 1 | 30.38 | 0.16 |
| 2 | 32.44 | 0.18 |
| 3 | 28.50 | 0.15 |
| 4 | 24.24 | 0.11 |
| 5 | 32.64 | 0.17 |
| 6 | 28.31 | 0.15 |
| 7 | 30.16 | 0.14 |
| 8 | 28.00 | 0.14 |
| 9 | 30.08 | 0.16 |

### CONCLUSION

The foregoing description of various embodiments of the invention has been presented for purposes of illustration and description. It is not intended to limit the invention to the precise forms disclosed. Many modifications, variations and refinements will be apparent to practitioners skilled in the art. For example, embodiments of the apparatus can be sized and otherwise adapted for various pediatric and neonatal applications as well as various veterinary applications (e.g., canine, feline, equine, bovine, ovine, porcine or other mammals).

Elements, characteristics, or acts from one embodiments can be readily recombined or substituted with one or more embodiments, characteristics or acts from other examples to form numerous additional embodiments within the scope of the invention. Moreover, elements that are shown or described as being combined with other elements, can, in various embodiments, exist as standalone elements. Also for any positive recitation of an element, characteristic, constituent, feature or step embodiments of the invention specifically contemplate the exclusion of the element, value, characteristic, constituent, feature, step or the like. Hence, the scope of the present invention is not limited to the specifics of the described examples, but is instead limited solely by the appended claims.

1. An apparatus for in vivo delivery of solid form medication within the body of a patient, the apparatus comprising:
a housing configured to be implanted within the body of the patient, the housing having a wall;
a spool rotatably disposed within the housing and configured to receive a flexible drug cord wound over an outer surface thereof;
a port in the housing wall configured for advancing the drug cord out of the housing;
a flexible seal on the port configured to seal over the drug cord as the drug cord is advanced through the port;
a drug cord advancement mechanism configured to advance the drug cord through the port and out of the housing; and
a controller configured to actuate the drug cord advancement mechanism to advance a length of the drug cord out of the housing in response to an input.

2. The apparatus of claim 1, further comprising a diffusion chamber configured to receive the length of the drug cord and expose the drug cord to body fluid to dissolve the drug cord and allow a solution comprising the drug to diffuse out of the diffusion chamber.

3. The apparatus of claim 1, further comprising cutting means associated with the housing, the cutting means configured to cut the cord after it has been dispensed out of the housing.

4. The apparatus of claim 3, wherein the cutting means comprise a blade.

5. The apparatus of claim 1, wherein the drug cord advancement mechanism includes driver elements which engage and push the drug cord and a motor coupled to the drive elements.

6. The apparatus of claim 5, wherein the driver elements comprise a pair of opposed pinch rollers.

7. The apparatus of claim 5, wherein the driver elements comprise a reciprocating linear advancement mechanism.

8. The apparatus of claim 2, further comprising a catheter coupled to the port and configured to guide the flexible drug cord to the diffusion chamber.

9. The apparatus of claim 8, wherein the diffusion chamber is coupled to a distal portion of the catheter.

10. The apparatus of claim 8, wherein the catheter has an atraumatic distal tip to allow for extended periods of implantation at an implantation site.

11. The apparatus of claim 1, wherein at least a portion of the apparatus includes a biocompatible coating.

12. The apparatus of claim 11, wherein the biocompatible coating comprises silicone, a polyurethane or a fluoropolymer.

13. The apparatus of claim 1, wherein the controller is configured to deliver a dose of medication at regular intervals.

14. The apparatus of claim 1, wherein the controller is configured to respond to a signal transmitted from outside the patient's body.

15. The apparatus of claim 1 wherein the controller is configured to respond to a signal received from a sensor within or on the patient's body.

16. The apparatus of claim 15, wherein the signal received from the sensor corresponds to a physiological event.

17. The apparatus of claim 16, wherein the physiological event is an epileptic seizure, a pre-seizure event, an arrhythmia, hyperglycemia or hypertension.

18. A flexible drug cord comprising:
a matrix material formed into an elongate, flexible cord, wherein the matrix material is degradable in a physiologic environment and non-degradable in a hermetically sealed environment;
at least one medicament dispersed within at least a length of the matrix material; and
wherein the matrix material formed into an elongate, flexible cord is configured to be: (i) wound and stored over a spool in a housing implanted within a patient's body for an extended period without substantial degradation or deleterious effect to the medication, (ii) delivered to a delivery site by unwinding from the spool and advancing through a port in the housing, and (iii) dissolved in tissue fluids at a target tissue site to release the medicament and produce a therapeutic effect at the target tissue site to treat a disease or condition.

19. The flexible drug cord of claim 18, wherein the flexible drug cord comprises a medicament for the treatment of epilepsy.

20. The flexible drug cord of claim 19, wherein the medicament comprises furosemide or an analogue or derivative of furosemide.

21. The flexible drug cord of claim 20, wherein the drug cord contains about 1% to about 10% by weight of furosemide or that of the analogue or derivative of top furosemide.

22. The flexible drug cord of claim 20, wherein the drug cord has an axial stiffness of about 9.60N/mm.

23. The flexible drug cord of claim 20, wherein the drug cord has a Young's modulus of about 958.0 Mpa.

24. The flexible drug cord of claim 18, wherein the medicament comprises a medicament for the treatment of an arrhythmia.

25. The flexible drug cord of claim 24, wherein the medicament comprises lidocaine, atropine or flecainide.

26. The flexible drug cord of claim 18, wherein the medicament comprises a medicament for the treatment of diabetes.

27. The flexible drug cord of claim 18, wherein the medicament comprises a medicament for the treatment of angina.

28. The flexible drug cord of claim 27, wherein the medicament comprises nitroglycerine.

29. The flexible drug cord of claim 18, wherein the medicament comprises a medicament for the treatment of cancer.

30. The flexible drug cord of claim 29, wherein the medicament comprises a medicament for the treatment of brain cancer or glioblastoma.

31. The flexible drug cord of claim 30, wherein the medicament comprises a medicament for the treatment of brain cancer or glioblastoma.

32. The flexible drug cord of claim 31, wherein the medicament is topotecan or an analogue or derivative of topotecan.

33. The flexible drug cord of claim 32, wherein the drug cord contains about 1% to about 10% by weight of topotecan or that of the analogue or derivative of topotecan.

34. The flexible drug cord of claim 31, wherein a dose of topotecan or the analogue or derivative of topotecan in the drug cord is in a range of about 0.01 to about 0.04 mg.

35. The flexible drug cord of claim 31, wherein the drug cord has an axial stiffness of about 9.01 N/mm.

36. The flexible drug cord of claim 31, wherein the drug cord has a Young's modulus of about 921 Mpa.

37. The flexible drug cord of claim 18, wherein the medicament comprises a plurality of medicaments.

38. The flexible drug cord of claim 18, wherein the medicament comprises a pharmaceutical excipient.

39. The flexible drug cord of claim 38, wherein the excipient comprise at least one of a disintegrant or a preservative.

40. A method for delivering medicament to a patient, the method comprising:
implanting an enclosure containing an elongate flexible drug cord wound on a spool in the body of a patient for an extended period without substantial degradation or deleterious effect to the medicament;
unwinding the spool to advance a length of the flexible drug cord through a port on a wall of the enclosure and to a tissue target site in the body of a patient; and
wherein the length of the flexible drug cord degrades to release the medicament at the tissue target site to produce a therapeutic effect for the treatment of a disease or condition.

41. The method of claim 40, wherein unwinding the spool to advance a length of the flexible drug cord comprises rotating pinch rollers to draw the length of the flexible drug cord from the spool and advance the length through a port on the enclosure.

42. The method of claim 40, wherein unwinding the spool to advance a length of the flexible drug cord comprises engaging the length of the flexible drug cord with a reciprocating linear driver to draw the length of the flexible drug cord from the spool and advance the length through a port on the enclosure.

43. The method of claim 40, wherein the length of the flexible drug cord is advanced into a diffusion chamber to expose the length of the drug cord to body fluid to dissolve the length of the drug cord and allow a solution comprising the medicament to diffuse out of the diffusion chamber to the tissue target site.

44. The method of claim 30, wherein the diffusion chamber is attached to the port on the wall of the enclosure by a catheter so that the tissue target site can be located remotely from a site of implantation of the enclosure.

45. The method of claim 40, further comprising cutting the flexible drug cord to dispense the length of the drug cord that degrades as a single dose of medicament.

46. The method of claim 40, wherein the extended period is up to about five years.

47. The method of claim 40, wherein the length of the flexible drug cord is delivered at regular intervals.

48. The method of claim 47, wherein the interval is selected from the group consisting of from one hour to seven days, from one hour to two days, from one hour to one day, one hour to 12 hours, from two hours to 12 hrs and from two hours to one day.

49. The method of claim 40, wherein the length of the flexible drug cord is delivered in response to a sensed physiological parameter.

50. The method of claim 49, wherein the sensed physiological parameter is predictive of at least one of a medical condition, a neurological seizure, an epileptic seizure, an arrhythmia, hypertension or hyperglycemia.

51. The method of claim 40, wherein the dose of medicament comprises a medicament for the treatment of epilepsy.

52. The method of claim 51 wherein the medicament comprises furosemide.

53. The method of claim 40, wherein the length of the flexible drug cord comprises a medicament for the treatment of an arrhythmia.

54. The method of claim 53, wherein the medicament comprises lidocaine, atropine or flecainide.

55. The method of claim 40, wherein the length of the flexible drug cord comprises a medicament for the treatment of diabetes.

56. The method of claim 55, wherein the medicament comprises insulin or an incretin.

57. The method of claim 40, wherein the length of the flexible drug cord comprises a medicament for the treatment of angina.

58. The method of claim 57, wherein the medicament comprises nitroglycerine.

59. The method of claim 40, wherein the length of the flexible drug cord comprises a medicament for the treatment cancer.

60. The method of claim 59, wherein the medicament comprises a medicament for the treatment of brain cancer or glioblastoma.

61. The method of claim 60, wherein the medicament is topotecan or an analogue or derivative of topotecan.

62. The method of claim 61, wherein the drug cord contains about 1% to about 10% by weight of topotecan or that of the analogue or derivative of topotecan.

63. The method of claim 61, wherein a dose of topotecan or the analogue or derivative of topotecan in the length of drug cord is in a range of about 0.01 to about 0.04 mg.
